(19) United States
(12) Patent Application Publication
(10) Pub. No.: US 2004/0254173 A1
(43) Pub. Date: Dec. 16, 2004

(54) Modulation of dopamine responses with substituted (S)-2,3-benzodiazepines

(76) Inventors: Steven M. Leventer, Langhorne, PA (US); Herbert W. Harris, Merion, PA (US); Robert F. Kucharik, Glenmoore, PA (US)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/461,290

(22) Filed: Jun. 13, 2003

(51) Int. Cl. A61K 31/5513

(52) U.S. Cl. 514/221

(57) ABSTRACT

Compounds according to formula I:

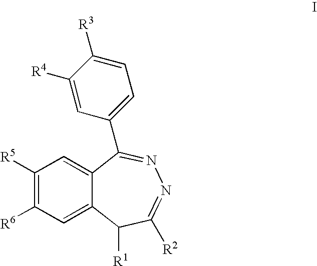

wherein R¹, R², R³, R⁴, R⁵ and R⁶ are as defined herein, and wherein the compound comprises the (S)-enantiomer, administered for modulation of dopamine responses and treatment of dopamine-mediated disorders.

### FIELD OF THE INVENTION

The present invention relates to methods of modulation of dopamine responses and treatment for dopamine-mediated disorders.

### BACKGROUND OF THE INVENTION

2,3-Benzodiazepines

Certain 2,3-benzodiazepines have been explored extensively for their potent CNS modulating activity. Compounds such as tofisopam (Grandaxin®) (structure shown below, with the atom numbering system indicated), girisopam, and norisopam have demonstrated substantial anxiolytic and antipsychotic activity.

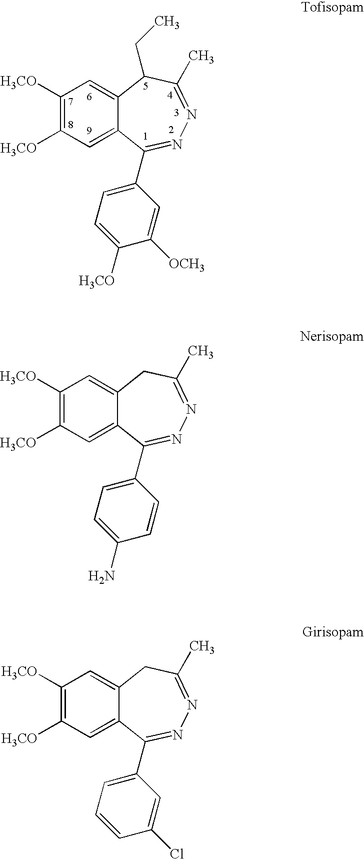

Tofisopam has been shown in humans to have an activity profile that is significantly different from that of widely used 1,4-benzodiazepine (BZ) anxiolytics such as diazepam (Valium®) and chlordiazepepoxide (Librium®). The 1,4-benzodiazepines, in addition to having sedative-hypnotic activity, also possess muscle relaxant and anticonvulsant properties which, though therapeutically useful in some disease states, are nonetheless potentially untoward side effects. Thus the 1,4-benzodiazepines, though safe when administered alone, may be dangerous in combination with other CNS drugs, including alcohol.

Tofisopam, in contrast, is a non-sedative anxiolytic that has no appreciable sedative, muscle relaxant or anticonvulsant properties See Horvath et al., Progress in Neurobiology, 60 (2000), 309-342, the entire disclosure of which is incorporated herein by reference. In clinical studies, tofisopam improved rather than impaired psychomotor performance and showed no interaction with ethanol (Id.). These observations comport with data that show that tofisopam does not interact with central BZ receptors and binds only weakly to peripheral BZ receptors.

Other 2,3-benzodiazepines that are structurally similar to tofisopam have been investigated and shown to have varying activity profiles. For example, GYKI-52466 and GYKI-53655 (structures shown below) act as noncompetitive glutamate antagonists at the AMPA (α-amino-3-hydroxy-5-methyl-4-isoxazolepropionic acid) site, and have demonstrated neuroprotective, muscle relaxant and anticonvulsant activity (Id.). Another group of 2,3-benzodiazepines that have been investigated are represented by the compound GYKI-52895, and show activity as selective dopamine uptake inhibitors with potential use in antidepressant and anti-Parkinsonism therapy.

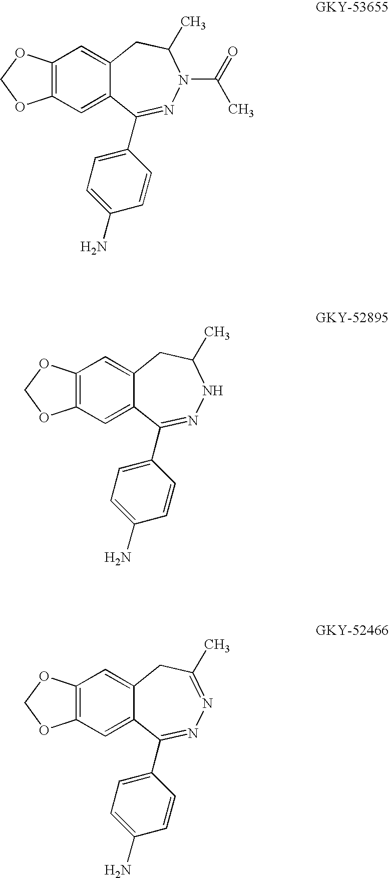

Tofisopam is a racemic mixture of (R)- and (S)-enantiomers. This is due to the asymmetric carbon, i.e., a carbon with four different groups attached, at the 5-position of the benzodiazepine ring.

The structure and conformational properties of tofisopam have been determined by NMR, CD and X-ray crystallography See Visy et al., Chirality 1:271-275 (1989), the entire disclosure of which is incorporated herein by reference. The 2,3-diazepine ring exists as two different conformers. The major conformers, (R)-(+) and (S)-(−) have the 5-ethyl group in a quasi-equatorial position. In the minor conformers, (R)-(−) and (S)-(+), the 5-ethyl group is positioned quasi-axially. Thus, racemic tofisopam may exist as four molecular species, i.e., two enantiomers, each of which exists in two conformations. The sign of the optical rotation is reversed upon inversion of the diazepine ring from one conformer to the other. In crystal form, tofisopam exists only as the major conformations, with dextrorotatory tofisopam being of the (R) absolute configuration. See Toth et al., J. Heterocyclic Chem., 20:709-713 (1983); Fogassy et al., Bioorganic Heterocycles, Van der Plas, H. C., Ötvös, L, Simongi, M., eds. Budapest Amsterdam: Akademia; Kiado-Elsevier, 229:233 (1984), the entire disclosures of which are incorporated herein by reference.

Differential binding of these two conformations of tofisopam is reported in binding studies with human albumin See Simongi et al. Biochem. Pharm., 32(12), 1917-1920, 1983, the entire disclosure of which is incorporated herein by reference. The two conformers have also been reported as existing in equilibrium See Zsila et al., Journal of Liquid Chromatography & Related Technologies, 22(5), 713-719, 1999; and references therein, the entire disclosures of which are incorporated herein by reference.

The optically pure (R)-enantiomer of tofisopam, (R)-1-(3,4-dimethoxyphenyl)-4-methyl-5-ethyl-7,8-dimethoxy-5H-2,3-benzodiazepine), has been isolated and shown to possess the nonsedative anxiolytic activity of the racemic mixture. See U.S. Pat. No. 6,080,736; the entire disclosure of which is incorporated herein by reference. The optically pure (S)-enantiomer of tofisopam, (S)-1-(3,4-dimethoxyphenyl)-4-methyl-5-ethyl-7,8-dimethoxy-5H-2,3-benzodiazepine) has also been isolated and shown to possess an anticonvulsant activity and antiseizure activity. See US Patent Publication No. US-2003-0055048-A1; the entire disclosure of which is incorporated herein by reference.

Metabolism of Tofisopam

Tofisopam is metabolized in human, rat, dog, monkey and rabbit to one or more of six major metabolites, depending on the host species. These metabolites have been identified as: 1-(3,4-dimethoxyphenyl)-4-methyl-5-ethyl-7-hydroxy-8-methoxy-5H-2,3-benzodiazepine; 1-(3,4-dimethoxyphenyl)-4-methyl-5-ethyl-7-methoxy-8-hydroxy-5H-2,3-benzodiazepine; 1-(3-methoxy-4-hydroxyphenyl)-4-methyl-5-ethyl-7,8-dimethoxy-5H-2,3-benzodiazepine; 1-(3-hydroxy-4-methoxyphenyl)-4-methyl-5-ethyl-7,8-dimethoxy-5H-2,3-benzodiazepine; 1-(3-methoxy-4-hydroxyphenyl)-4-methyl-5-ethyl-7-hydroxy-8-methoxy-5H-2,3-benzodiazepine; and 1-(3-hydroxy-4-methoxyphenyl)-4-methyl-5-ethyl-7-hydroxy-8-methoxy-5H-2,3-benzodiazepine. See Tomori et al., Journal of Chromatography, 241 (1982), p. 89-99. Of these six metabolites of tofisopam, 1-(3,4-dimethoxyphenyl)-4-methyl-5-ethyl-7-hydroxy-8-methoxy-5H-2,3-benzodiazepine; 1-(3-methoxy-4-hydroxyphenyl)-4-methyl-5-ethyl-7,8-dimethoxy-5H-2,3-benzodiazepine; and 1-(3-methoxy-4-hydroxyphenyl)-4-methyl-5-ethyl-7-hydroxy-8-methoxy-5H-2,3-benzodiazepine have been identified as human metabolites. These three human metabolites have been synthesized and tested in certain pharmacological assays. See C. Ito, “Behavioral Pharmacological Study on the Structure Activity Relationship of Benzodiazepine Derivatives: With Particular Reference to the Activity of 2,3-Benzodiazepine,” J. Tokyo Med. College, 39:369-384 (1981).

The three metabolites were tested in an assay of inhibition of aggression in mice; an assay of muricide (mouse killing behavior) in rats; and In assays testing for anti-noradrenergic effects. 1-(3,4-Dimethoxyphenyl)-4-methyl-5-ethyl-7-hydroxy-8-methoxy-5H-2,3-benzodiazepine showed 0% inhibition of aggression in mice; showed 20% inhibition of muricide; and showed no antinoradrenergic effect. 1-(3-Methoxy-4-hydroxyphenyl)-4-methyl-5-ethyl-7,8-dimethoxy-5H-2,3-benzodiazepine showed 0% inhibition of aggression in mice; 0% inhibition of muricide; and a measurable antinoradrenergic effect. 1-(3-Methoxy-4-hydroxyphenyl)-4-methyl-5-ethyl-7-hydroxy-8-methoxy-5H-2,3-benzodiazepine showed 28.6% inhibition of aggression in mice; 20% inhibition of muricide; and a measurable antinoradrenergic effect. See Ito, Id.

1-(3,4-Dimethoxyphenyl)-4-methyl-5-ethyl-7-hydroxy-8-methoxy-5H-2,3-benzodiazepine; 1-(3-methoxy-4-hydroxy-phenyl)-4-methyl-5-ethyl-7,8-dimethoxy-5H-2,3-benzodiazepine; 1-(3-methoxy-4-hydroxyphenyl)-4-methyl-5-ethyl-7-hydroxy-8-methoxy-5H-2,3-benzodiazepine; and 1-(3-hydroxy-4-methoxyphenyl)-4-methyl-5-ethyl-7-hydroxy-8-methoxy-5H-2,3-benzodiazepine are also disclosed in U.S. Pat. No. 4,322,346, the entire disclosure of which is incorporated herein by reference. 1-(3-Methoxy-4-hydroxy-phenyl)-4-methyl-5-ethyl-7,8-dimethoxy-5H-2,3-benzodiazepine is reported therein to demonstrate narcosis-potentiating activity in mice.

Reported Causes of Seizures and Convulsions

Convulsions and seizures are symptoms of a disorder of the central nervous system, the most common of which is epilepsy. Seizures and convulsions are episodic rather than continuous, such that an individual suffering therefrom, will have periodic seizures. Treatments for convulsions and seizures with anticonvulsant drugs can prevent seizure activity by altering neurotransmitter activity in nerve cells, but cannot correct the underlying condition.

Many abnormalities of the nervous system can result in seizure activity. Seizures can also occur in the normal nervous system when its metabolic balance is disturbed. Some persons may have a genetic predisposition to the development of seizures. Seizures may develop at or around the time of a head injury or a stroke or may develop years after. Disorders that change levels of various metabolic substances in the body sometimes result in seizures, e.g., electrolyte imbalance, hypoglycemia, hyperglycemia, renal failure with uremia or changes that occur around the time of kidney dialysis, hepatic failure and associated elevation of toxins, hypoxia. Overdose of and abrupt withdrawal from some prescription drugs can result in seizure activity. Substances that may induce seizures include: tricyclic antidepressants, lithium, antipsychotic medications, aminophylline, and high doses of penicillin. Chronic illicit drug use also may cause seizures, particularly use of cocaine, heroine, amphetamines, and PCP. Alcohol withdrawal can produce seizures. Poisoning from carbon monoxide, lead, and other heavy metals also may cause seizures. Infections of the nervous system, including meningitis, encephalitis and human immunodeficiency virus (HIV) and related infections may result in seizure activity. Malignant and benign brain tumors may be associated with seizures. Several neurodegenerative disorders produce seizure activity, including: Alzheimer's disease, Creutzfeld-Jakob disease, neurofibromatosis, phenylketonuria (PKU), tuberous sclerosis, Sturge-Weber syndrome, Tay-Sachs disease, and cerebral palsy.

Seizures may be initiated by an identifiable “trigger.” Triggers do not cause seizures but provoke the onset of a seizure or cause a seizure in a patient wherein the underlying disorder such as epilepsy is under control. Alcohol consumption, hormonal changes of the menstrual cycle, sleep deprivation, flickering or flashing light, and stress can trigger a seizure in a susceptible person.

Dopamine-Mediated Disorders

Dopamine is a catecholamine neurotransmitter. It is derived from tyrosine and is the precursor to norepinephrine and epinephrine. Dopamine is a major transmitter in the extrapyramidal system of the brain, and important in regulating movement. It is found in neurons of both the central and peripheral nervous systems. It is stored in vesicles in axon terminals and released when the neuron is depolarized. Dopamine interacts with specific membrane receptors to produce its effects. Removal of dopamine from the synapse by reuptake of dopamine into the presynaptic neuron or by metabolic inactivation by monoamine oxidase B (MAO-B) or catechol-O-methyltransferase (COMT) terminates dopamine effects.

Activation of the striatal D₂ dopamine receptor subfamily in rats results in a behavioral syndrome known as stereotypy, made up of repetitive sniffing and gnawing, accompanied by an increase in the animals' activity. The repetitive behaviors observed in people following amphetamine ingestion may have a similar neurochemical basis. Blockade of the striatal D₂ receptor subfamily produces increases in muscle rigidity in rats and a Parkinson-like syndrome in humans. In both rats and humans, administration of a D₂ antagonist results in blocking of dopamine's inhibition of prolactin release, and thus an increase in prolactin release from the anterior pituitary.

Drugs affecting dopamine transmission may act in several ways. Many drugs affect dopamine transmission directly by either blocking or stimulating its receptors. For example, antipsychotic drugs are dopamine antagonists, whereas bromocriptine, used to treat hyperprolactinaemia and Parkinson's disease, is a dopamine agonist.

Several drugs of clinical importance act indirectly to modify dopamine levels. Levodopa, for example, is converted to dopamine, and amphetamine serves to release dopamine from terminal stores. Other drugs increase the synaptic concentration of dopamine by blocking its uptake or metabolism. For example, the addictive properties of cocaine may be due to its potent inhibition of dopamine re-uptake. In contrast, selegiline, a MAO-B inhibitor, elevates dopamine concentrations by inhibiting its breakdown.

Two primary pathways of dopamine action demonstrate involvement in pathological processes. First, degeneration of the dopaminergic neurons of the nigrostriatal pathway, the region involved in the control of motor function, is associated with the motor symptoms of Parkinson's disease, i.e. bradykinesia, tremor and rigidity. Second, overactivity of dopamine neurotransmission in the mesolimbic and mesocortical pathways, regions associated with cognition and emotionality, may underlie the positive symptoms of schizophrenia, i.e. thought disorder, delusions and hallucinations.

Currently, the management of many psychiatric and movement disorders relies heavily on the inhibition or facilitation of dopamine at its receptors. Via dopamine receptors, dopamine and dopamine mimetic ligands, such as antipsychotic drugs, exert both short and long term changes in ion channel activity, protein kinase/phosphatase activities, and gene expression (Artalejo et al., Nature 348:239-42 (1990); Steiner and Gerfen, J. Comp. Neurol. 353:200-12 (1995); Surmeirer et al., Neuton 14:385-97 (1995)).

Peripheral dopamine receptors mediate a variety of effects including changes in blood flow, glomerular filtration rate, sodium excretion, catecholamine release and inotropic effects on the heart.

When dopamine-containing neurons in the central nervous system undergo degeneration, there is an interference of normal synaptic transmission. This interference is characterized by a depletion of functional dopamine, accompanied by a change in the number and affinity of dopamine receptors. This results in decreased synaptic neurotransmission. Various neurological and neuropsychiatric disorders such as Parkinsonism are currently viewed as resulting from depletion of brain dopamine.

What are needed are new agents to mediate dopamine transmission.

### SUMMARY OF THE INVENTION

In one embodiment of the invention a method of modulating dopamine responses in the central nervous system of an individual, is provided, comprising administering to the individual an effective amount of at least one compound of formula I:

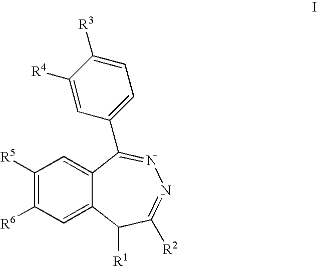

wherein:

R¹ is —(C₁-C₇)hydrocarbyl or —(C₂-C₆)heteroalkyl;

R² is —H or —(C₁-C₇)hydrocarbyl; wherein R¹ and R² may combine to form a carbocyclic or heterocyclic 5- or 6-membered ring; and

R³, R⁴, R⁵ and R⁶ (hereinafter, collectively “phenyl ring substituents”) are independently selected from the group consisting of —OH, —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen, wherein R⁵ and R⁶ may combine to form a 5,6- or 7-membered heterocyclic ring; or a pharmaceutically acceptable salt thereof;

wherein the administered compounds according to formula I comprise an (S)-enantiomer, substantially free of the corresponding (R)-enantiomer of the same compound.

The (S)-enantiomer of a compound of formula I is a compound that is in the (S) absolute configuration with respect to the 5-position of the benzodiazepine ring.

In another embodiment of the invention, a method of treating a dopamine-mediated disorder in an individual not suffering from seizures or convulsions is provided comprising administering to the individual an effective amount of at least one compound according to formula I as described above.

In another embodiment of the invention, a method of treating a dopamine-mediated disorder in an individual is provided comprising administering to the individual, an effective amount of at least one compound according to formula I as described above;

provided that the dopamine-mediated disorder is not one which causes seizures or convulsions.

According to some embodiments of the invention, all of R³, R⁴, R⁵ and R⁶ are independently selected from —O(C₁-C₇)hydrocarbyl, preferably —O(C₁-C₇)alkyl, most preferably —OCH₃.

One preferred compound for use in the practice of the invention is (S)-1-(3,4-dimethoxyphenyl)-4-methyl-5-ethyl-7,8-dimethoxy-5H-2,3-benzodiazepine, or a pharmaceutically-acceptable salt thereof.

According to other embodiments of the invention, one of R³, R⁴, R⁵ or R⁶ is —OH, and the remaining phenyl ring substituents are independently selected from the group consisting of —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen, wherein R⁵ and R⁶ may combine to form a 5,6- or 7-membered heterocyclic ring; or a pharmaceutically acceptable salt thereof.

According to a first sub-embodiment of the invention, one of R³ or R⁴ is —OH, and the other phenyl ring substituents are independently selected from the group consisting of —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen.

According to a second sub-embodiment of the invention, one of R³ or R⁴ is —OH, one or two of the other phenyl ring substituents is —OCH₃, and the other phenyl ring substituent(s) are independently selected from the group consisting of —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen.

According to a third sub-embodiment of the invention, one phenyl ring substituent is —OH, and the other phenyl ring substituents are independently selected from —O(C₁-C₇)hydrocarbyl, preferably —O(C₁-C₇)alkyl, most preferably —OCH₃.

In some embodiments of the invention, R¹ and R² are independently selected from —(C₁-C₇)alkyl, preferably, —(C₁-C₃)alkyl. In a preferred sub-embodiments R¹ is —CH₂CH₃ and R² is —CH₃.

Other preferred compounds for use in the practice of the invention, are selected from the group consisting of:

(S)-1-(3,4-dimethoxyphenyl)-4-methyl-5-ethyl-7-hydroxy-8-methoxy-5H-2,3-benzodiazepine;

(S)-1-(3-hydroxy-4-methoxyphenyl)-4-methyl-5-ethyl-7,8-dimethoxy-5H-2,3-benzodiazepine;

(S)-1-(3-methoxy-4-hydroxyphenyl)-4-methyl-5-ethyl-7,8-dimethoxy-5H-2,3-benzodiazepine;

(S)-1-(3,4-dimethoxyphenyl)-4-methyl-5-ethyl-7-methoxy-8-hydroxy-5H-2,3-benzodiazepine;

or pharmaceutically-acceptable salts thereof.

Most preferred for use in the practice of the invention, is the compound,

(S)-1-(3,4-dimethoxy)-4-methyl-5-ethyl-7,8-dimethoxy-5H-2,3-benzodiazepine;

or a pharmaceutically-acceptable salt thereof.

### Definitions

The expression “dopamine-mediated disorder” refers to a disorder, either chronic or recurrent, characterized by abnormality in dopaminergic neurotransmission.

The expression “neurological disorder” refers to disorders wherein there is a malfunction of the nervous system, including, for example, cerebral palsy, epilepsy, and Parkinsonism.

The expression “neuropsychiatric disorder,” refers to a disorder that affects the mental state of an individual.

The expression “modulating dopamine responses” refers to the direct or indirect modification of synaptic neurotransmission involving the neurotransmitter dopamine. The modulation may comprise agonism of a dopamine response, i.e., increasing a dopamine response. Alternatively, the modulation may comprise antagonism, i.e., decreasing a dopamine response.

As used herein the expressions “Parkinson's disease”, “Parkinson's” and “Parkinsonism” are inclusive of the various forms of the condition including idiosyncratic Parkinson's disease, post-encephalitic Parkinson's disease, drug induced Parkinson's disease, such as neuroleptic induced Parkinsonism, and post-ischemic Parkinsonism.

The term “convulsion” means a violent involuntary contraction or series of contractions of the voluntary muscles.

The term “seizure” refers to a sudden attack or convulsion due to involuntary electrical activity in the brain. A seizure can result in a wide variety of clinical manifestations such as, for example, muscle twitches, staring, tongue biting, urination, loss of consciousness and total body shaking.

The term “acyl” means a radical of the general formula —C(═O)—R, wherein —R is hydrogen, hydrocarbyl, amino, alkylamino, dialkylamino hydroxy or alkoxy.” Examples include for example, acetyl (—C(═O)CH₃), propionyl (—C(═O)CH₂CH₃), benzoyl (—C(═O)C₆H₅), phenylacetyl (—C(═O)CH₂C₆H₅), carboethoxy (—CO₂CH₂CH₃), and dimethylcarbamoyl (—C(═O)N(CH₃)₂). When the R group in the acetyl radical is alkoxy, alkyl amino or dialkyl amino, the alkyl portion is preferably (C₁-C₆)alkyl, more preferably (C₁-C₃)alkyl. When the R is hydrocarbyl, it is preferably (C₁-C₇)hydrocarbyl. When R is hydrocarbyl, it is preferably alkyl, more preferably (C₁-C₇)alkyl.

The term “alkyl”, by itself or as part of another substituent means, unless otherwise stated, a straight, branched or cyclic chain hydrocarbon radical, including di- and multi-radicals, having the number of carbon atoms designated (i.e. C₁-C₆ means one to six carbons). Alkyl groups include straight chain, branched chain or cyclic groups, with straight being preferred. Examples include: methyl, ethyl, propyl, isopropyl, butyl, isobutyl, tert-butyl, pentyl, neopentyl, hexyl, cyclohexyl and cyclopropylmethyl. (C₁-C₆)alkyl is preferred. Most preferred is (C₁-C₃)alkyl, particularly ethyl, methyl and isopropyl.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred is (C₁-C₆)alkoxy. More preferred is (C₁-C₃)alkoxy, particularly ethoxy and methoxy.

The term “amine” or “amino” refers to radicals of the general formula —NRR′, wherein R and R′ are independently selected from hydrogen or a hydrocarbyl radical, or wherein R and R′ combined form a heterocycle. Examples of amino groups include: —NH₂, methyl amino, diethyl amino, anilino, benzyl amino, piperidinyl, piperazinyl and indolinyl. Preferred hydrocarbyl radicals are (C₁-C₇)hydrocarbyl radicals. More preferred are hydrocarbyl radicals that are (C₁-C₇)alkyl radicals.

The term “aromatic” refers to a carbocycle or heterocycle having one or more polyunsaturated rings having aromatic character (4n+2) delocalized π (pi) electrons).

The term “aryl” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl.

The term “hydrocarbyl” refers to any moiety comprising only hydrogen and carbon atoms. This definition includes for example alkyl, alkenyl, alkynyl, aryl and benzyl groups. Preferred are (C₁-C₇)hydrocarbyl.

The term “heteroalkyl” by itself or in combination with another term means, unless otherwise stated, a stable straight or branched chain radical consisting of the stated number of carbon atoms and one or two heteroatoms selected from the group consisting of O, N, and S. Nitrogen and sulfur atoms may be optionally oxidized to the N-oxide and sulfoxide or sulfone, respectively. In addition, a nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Preferred are (C₂-C₆)heteroalkyl. More preferred are (C₂-C₄)heteroalkyl. Examples include: —O—CH₂—CH₂—CH₃, —CH₂—CH₂CH₂—OH, —CH₂—CH₂—NH—CH₃, —CH₂—C(═O)—CH₃, —CH₂—N═N—CH₂—CH₃, —CH₂—S—CH₂—CH₃, —CH₂CH₂—S(═O)—CH₃ and —CH₂—CH₂—NH—SO₂—CH₃. Up to two heteroatoms may be consecutive, such as, for example, —CH₂—NH—OCH₃, or —CH₂—CH₂—S—S—CH₃. More preferred are heteroalkyl groups containing one or two oxygen atoms.

When two groups combine to form a carbocyclic 5- or 6-membered ring, the ring is preferably saturated. Preferred heterocyclic rings are saturated rings containing one or two heteroatoms selected from N, O and S. Heterocyclic rings annulated to the benzodiazepine seven-membered ring in this way include, for example, furan, dihydrofuran, tetrahydrofuran, pyran, dihydropyran, tetrahydropyran, thiophene, dihydrothiophene, tetrahydrothiophene, pyrrole, dihydropyrrole, pyrrolidine, pyridine, dihydropyridine, tetrahydropyridine and piperidine.

When two groups combine to form a 5-, 6- or 7-membered heterocyclic ring, preferred heterocyclic rings are 5- or 6-membered rings containing one or two heteroatoms selected from N, O and S. More preferred are heterocyclic rings containing one heteroatom selected from N and S. Most preferred are heterocyclic rings containing two oxygen atoms. Heterocyclic rings annulated to the benzodiazepine phenyl ring in this way include, for example, furan, dihydrofuran, dioxane, dioxolane, pyran, dihydropyran, tetrahydropyran, thiophene, dihydrothiophene, pyridine, dihydropyridine, tetrahydropyridine, piperidine, pyrrole, dihydropyrrole, imidazole, dihydroimidazole, thiazole, dihydrothiazole, oxazole, and dihydrooxazole.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position.

The expression “optically active” refers to a property whereby a material rotates the plane of plane-polarized light. A compound that is optically active is nonsuperimposable on its mirror image. The property of nonsuperimposablity of an object on its mirror image is called chirality.

The property of “chirality” in a molecule may arise from any structural feature that makes the molecule nonsuperimposable on its mirror image. The most common structural feature producing chirality is an asymmetric carbon atom, i.e., a carbon atom having four nonequivalent groups attached thereto.

The term “enantiomer” refers to each of the two nonsuperimposable isomers of a pure compound that is optically active. Single enantiomers are designated according to the Cahn-Ingold-Prelog system, a set of priority rules that rank the four groups attached to an asymmetric carbon. See March, Advanced Organic Chemistry, 4^{th} Ed., (1992), p. 109. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example below, the Cahn-Ingold-Prelog ranking sequence id A>B>C>D. The lowest ranking atom, D is oriented away from the viewer.

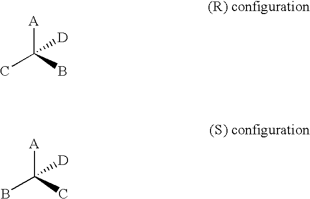

The term “racemate” or the phrase “racemic mixture” refers to a 50-50 mixture of two enantiomers such that the mixture does not rotate plane-polarized light.

The expression “enantiomeric excess,” generally reported as a percentage, is a means of expressing the degree of enantiomeric purity of a non-racemic mixture, i.e., a resolved or partially resolved enantiomer. The percent enantiomeric excess (% e.e.) is defined as:

$$\% enantiomeric excess = \frac{[ R ] - [ S ]}{[ R ] + [ S ]} \times 100 = \% R - \% S$$

The expression “(S)-enantiomer, substantially free of the (R)-enantiomer” means a composition that comprises at least about 80% e.e. of the (S)-enantiomer of a compound of formula I. Preferably, such a composition comprises at least about 90% e.e. of the (S)-enantiomer of a compound of formula I. More preferably, such a composition comprises at least about 95% e.e. of the (S)-enantiomer of a compound of formula I. Most preferably, such a composition comprises greater than 98% e.e. of the (S)-enantiomer of a compound of formula I.

The expression “effective amount” when used to describe the amount of drug administered to a patient suffering from a dopamine-mediated disorder, refers to an amount of a compound that alters (i.e., increases or decreases) dopamine-mediated neurotransmission, and thereby prevents or alleviates the symptoms of the disorder, when administered to a patient suffering from a disorder which results from abnormal dopaminergic neurotransmission.

The term “individual” or “subject” includes human beings and non-human animals. With respect to the disclosed methods of treating dopamine-mediated disorders, the terms “individual” and “subject” refer, unless the context indicates otherwise, to an organism that is afflicted with or diagnosed with such a disorder.

The term “treatment” includes measures taken with regard to an existing dopamine-mediated disorder and measures taken to prevent or delay the onset of a dopamine-mediated disorder.

The expressions “prevent” and “delay the onset” refer unless the context indicates otherwise, to an organism that has a medical history of dopamine-mediated disorder involving recurrent symptoms, or to an organism that has an increased likelihood of developing a dopamine-mediated disorder, e.g. by having hereditary or other risk factors for a dopamine-mediated disorder.

### DETAILED DESCRIPTION OF THE INVENTION

The compounds of formula I, and the pharmaceutically acceptable salts thereof, are useful as dopaminergic agents, i.e., they possess the ability to alter dopamine-mediated neurotransmission in mammals, including humans. They are therefore able to function as therapeutic agents in the treatment of a variety of conditions in mammals, the treatment of which can be effected or facilitated by an increase or decrease in dopamine-mediated neurotransmission.

Neurological disorders associated with altered dopamine function include movement disorders and cognitive disorders.

Movement disorders include, for example, Huntington's chorea; periodic limb movement syndrome; restless leg syndrome (akathesia); hyperkinesias, such as, for example, tardive dyskinesia; Tourrette's syndrome; Pick's disease; punch drunk syndrome; progressive subnuclear palsy; Parkinson's disease; multiple systems atrophy (Parkinson's plus); Landau-Kleffner syndrome; benign essential blepharospasm; amyotrophic lateral sclerosis (ALS); and medication-induced movement disorders such as, for example, neuroleptic-induced Parkinsonism, neuroleptic malignant syndrome, acute dystonia and extrapyramidal side effects from neuroleptic agents.

Cognitive disorders, include, for example, learning disorders; memory disorders, Alzheimer's disease and dementia, including, for example, pseudo dementia, hydrocephalic dementia, subcortical dementia and dementia due to Huntington's chorea or Parkinson's disease.

Neuropsychiatric disorders associated with altered dopamine function include, for example, psychosis; personality disorders; psychiatric mood disorders; conduct and impulse disorders; schizophrenia; bipolar disorders; dysphoric mania; attention deficit hyperactivity disorder (ADHD); depression; panic disorder; panic attacks; agoraphobia; obsessive-compulsive disorder; anxiety disorders such as, for example, post traumatic stress disorder, acute stress disorder, social anxiety disorder and generalized anxiety disorder; and eating disorders such as, for example, anorexia cachexia and anorexia nervosa.

In one embodiment of the invention, there is provided a method of treating a dopamine-mediated disorder in an individual provided that the dopamine-mediated disorder is not one which causes seizures or convulsions. Some neurological and neuropsychiatric disorders have been reported to be capable of causing seizures or convulsions.

Neurological disorders that are associated with altered dopamine function but have not been reported to be associated with convulsions or seizures include movement disorders and cognitive disorders.

Movement disorders associated with altered dopamine function and not reported as associated with convulsions or seizures include, for example, periodic limb movement syndrome; restless leg syndrome (akathesia); hyperkinesias such as, for example, tardive dyskinesia; Pick's disease; punch drunk syndrome; progressive subnuclear palsy; multiple systems atrophy (Parkinson's plus); Landau-Kleffner syndrome; benign essential blepharospasm; and medication-induced movement disorders such as, for example, neuroleptic malignant syndrome, acute dystonia, and extrapyramidal side effects from neuroleptic agents.

Cognitive disorders associated with altered dopamine function and not reported as associated with convulsions or seizures include, for example, learning disorders; memory disorders; and dementia, including, for example, pseudo dementia, hydrocephalic dementia, and subcortical dementia.

Neuropsychiatric disorders associated with altered dopamine function, but not reported to be associated with convulsions and seizures, include, for example, psychosis; personality disorders; psychiatric mood disorders; conduct and impulse disorders; bipolar disorders; dysphoric mania; attention deficit hyperactivity disorder (ADHD); depression; panic disorder; panic attacks; agoraphobia; anxiety disorders such as, for example, post traumatic stress disorder, acute stress disorder, social anxiety disorder and generalized anxiety disorder; and eating disorders such as, for example, anorexia cachexia and anorexia nervosa.

The compounds of formula I useful in the methods of the present invention may be prepared by one of several methods. These methods generally follow the synthetic strategies and procedures used in the synthesis of 2,3-benzodiazepines such as tofisopam and tofisopam analogs. See U.S. Pat. Nos. 3,736,315 and 4,423,044 (tofisopam syntheses) and Horvath et al., Progress in Neurobiology 60(2000) p.309-342 and references cited therein (preparation of tofisopam and analogs thereof), the disclosures of which are incorporated herein by reference. In the synthesis methods that follow, the products of the chemical synthesis are racemic compounds of formula I. The racemic mixture may be subsequently separated using known methods of resolution to produce the (S)-enantiomer substantially free of the corresponding (R)-enantiomer.

2,3-Benzodiazepines of formula I may be synthesized from the corresponding 2-benzopyrilium salt H by reaction with hydrazine hydrate, wherein X⁻ is a counterion such as, for example perchlorate:

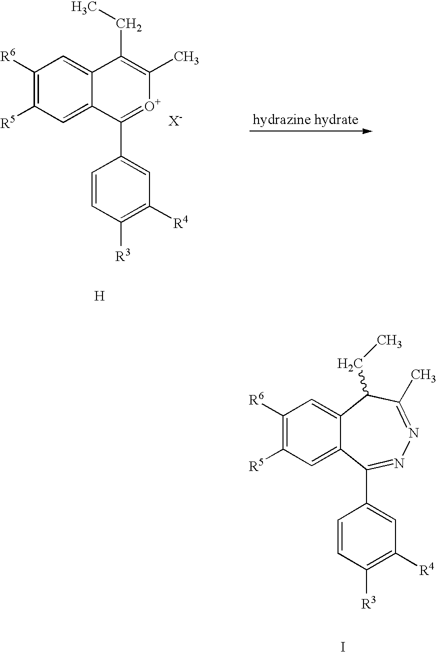

Accordingly, hydrazine hydrate (98%, approximately 3 equivalents based on the 2-benzopyrylium salt) is added dropwise to a stirred solution of the 2-benzopyrylium salt H in glacial acetic acid (approximately 1 mL/3 mmol of 2-benzopyrylium salt). During this operation, the solution is maintained at an elevated temperature, preferably, 80-100° C. The solution is then maintained a higher elevated temperature, preferably 95-100° C., for about one hour. Then the reaction mixture is diluted with 2% aqueous sodium hydroxide solution (approximately 3 equivalents based on the 2-benzopyrylium salt) and cooled. The product 2,3-benzodiazepine separates as a solid and is removed by filtration, washed with water and dried. The crude product may be purified by taking it up in a polar aprotic solvent such as dimethylformamide (DMF) at an elevated temperature, preferably 100-130° C., and decolorizing the solution with activated carbon. The carbon is removed by filtration and the filtered solution is diluted with water. The purified product precipitates out of the solution and is collected by filtration.

See Kórósi et al., U.S. Pat. No. 4,322,346, the entire disclosure of which is incorporated herein by reference, disclosing three variations of the reaction protocol for preparing a substituted 2,3-benzodiazepine from the precursor benzopyrilium salt.

Retrosynthetically, the intermediate benzopyrilium salt, H, may be prepared from one of several starting materials. According to one such method, illustrated in Scheme 1, intermediate H is prepared from the corresponding aryl ethanol derivative D via the isochroman intermediate F; wherein X⁻ is a counterion such as, for example, perchlorate:

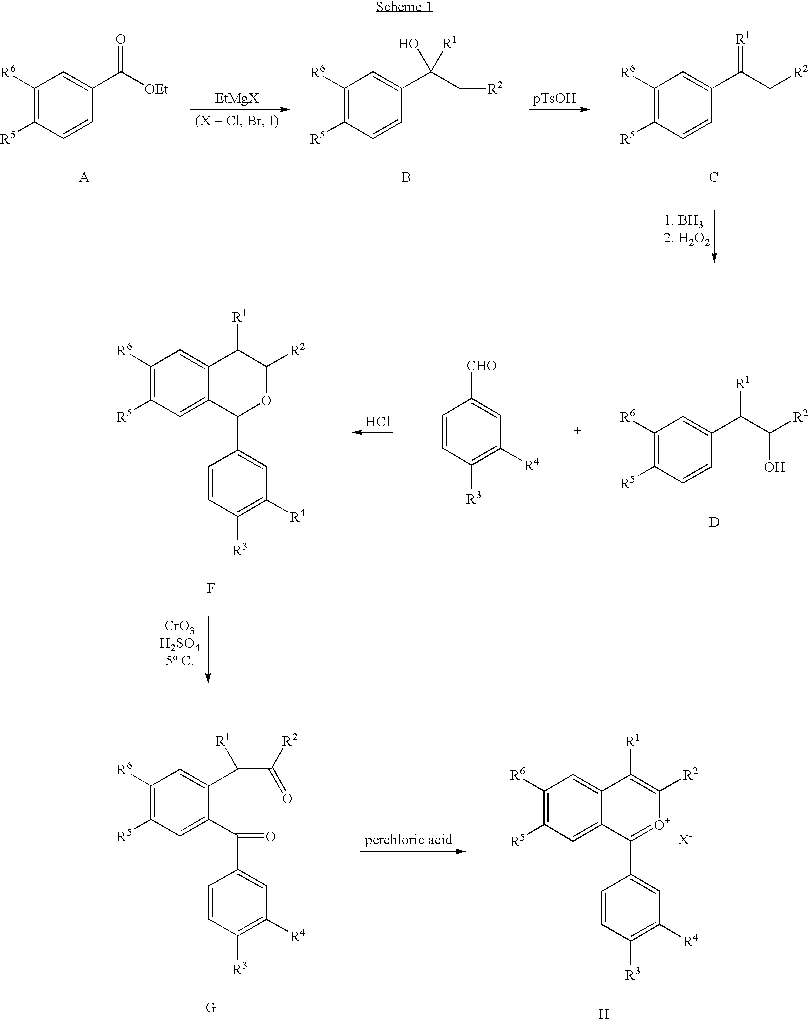

According to Scheme 1,3,4-disubstituted ethylbenzoate, A is dissolved in a suitable solvent, preferably ether and cooled to 0° C. Two equivalents of a selected Grignard reagent are added dropwise and the reaction is allowed to warm to room temperature and monitored for disappearance of starting material. When the reaction is complete, it may be quenched with a proton source such as acetic acid. Volatiles are removed in vacuo, and the product B is used for the next step without purification.

The α-substituted benzyl alcohol, B, is taken up in a high boiling solvent such as toluene and a catalytic amount of para-toluene sulfonic acid (p-TsOH). The mixture is warmed to reflux and may be monitored for disappearance of starting materials. When the reaction is complete, the volatiles are removed in vacuo and the crude product C is purified by column chromatography.

The substituted styrene, C is hydroxylated under anti-Markovnikov conditions to give intermediate phenylethyl alcohol D. A solution of D, and of a substituted benzaldehyde, E (1.2 eq) are dissolved in anhydrous dioxane. The resulting solution is then saturated with gaseous HCl and warmed, preferably to reflux temperature for about one hour. The mixture is then cooled to room temperature, poured into water, basified, preferably with aqueous sodium hydroxide and extracted with an organic solvent, preferably ethyl acetate. The extract is dried, filtered and concentrated under vacuum. The resulting residue is purified, preferably by crystallization to yield F.

To a stirred, cooled, (preferably to 0-5° C.) solution of F (2 g) in acetone (30 mL), is added dropwise a solution of chromium trioxide (2 g) in 35% sulfuric acid (20 mL); added at a rate such that the reaction temperature remains below 5° C. After the addition is complete, the reaction mixture is allowed to rise to room temperature and is stirred at room temperature for two hours. The reaction mixture is then poured into water and extracted with an organic solvent, preferably ethyl acetate. The organic extract is washed with water and then with ice-cold 10% aqueous sodium hydroxide. The aqueous alkaline fraction is then acidified, preferably with dilute aqueous hydrochloric acid and extracted with an organic solvent, preferably, chloroform. The chloroform extract is dried, filtered and concentrated under vacuum to give G. The crude residue may further be purified by column chromatography.

The 2-α-acyl hydrocarbylbenzophenone, G (5 g) is dissolved in glacial acetic acid (15 mL). To this mixture is added 60% perchloric acid (7.5 mL). The resulting mixture is warmed to 100° C. (steam bath) for three minutes. The mixture is allowed to cool to room temperature. Crystallization of the crude product may begin spontaneously at this point or may be induced by addition to the reaction mixture of ether or ethyl acetate. The product 2-benzopyrylium salt H is removed by filtration and purified by recrystallization, preferably from ethanol or glacial acetic acid/ethyl acetate.

A synthetic sequence, similar to that outlined above, for preparation of 2,3-benzodiazepines is disclosed in U.S. Pat. No. 3,736,315, the entire disclosure of which is incorporated herein by reference. Synthetic strategies for preparation of 2,3-benzodiazepines are also disclosed in Horvath et al., Progress in Neurobiology 60(2000) p309-342 and references cited therein; the entire disclosures of which are incorporated herein by reference.

Alternative methods for preparation of intermediate H start with an aryl acetonide or indanone starting material. See Kunnetsov, E. V., and Dorofeenko, G. N., Zh. Org Khim., 6, 578-581; and M. Vajda, Acta Chem. Acad. Sci. Hung, 40, p.295-307, 1964, respectively. Another variation for preparing 2,3-benzodiazepines is illustrated in Scheme 2 and 3 (Examples 1 and 2). The synthesis proceeds from intermediate G without isolation of the intermediate benzopyrilium salt H.

Resolution of 5-substituted-2,3-benzodiazepines to isolate (S)-enantiomers of formula I.

The synthetic procedures shown (or referenced) above result in racemic 2,3-benzodiazepines containing the (S)-enantiomers of formula I as a mixture with the corresponding (R)-enantiomer. The racemate must be resolved in order to isolate the (S)-enantiomer of formula I. Enantiomeric resolution may be achieved by converting racemic compositions to a pair of diastereomers by either covalently bonding to an optically active moiety, or by salt formation with an optically active base or acid. Either of these two methods provides a molecule with a second chiral center, thus generating a pair of diastereomers. This diastereomeric pair is then separated by conventional methods such as for example, crystallization or chromatography.

Racemic mixtures of compounds of formula I with their corresponding (R)-enantiomers may be converted to the (S)-dibenzoyltartaric acid salt, which is a diastereomeric mixture of (S,S) and (R,S) configurations. The pair of diastereomers (R,S) and (S,S) possess different properties, e.g., differential solubilities, that allow for the use of conventional separation methods. Fractional crystallization of diastereomeric salts from a suitable solvent is one such separation method. This resolution has been successfully applied to the resolution of racemic tofisopam. See Hungarian Patent 178516 and also Toth et al., J. Heterocyclic Chem., 20:09-713 (1983), the entire disclosures of which are incorporated herein by reference.

Alternatively, racemic mixtures of compounds of formula I with their corresponding (R)-enantiomers may be derivatized via, for example, acylation of the aromatic hydroxy moiety with a chiral acylating reagent such as, for example, (S)-mandelic acid. The resulting ester, has a second chiral center, and thus exists as a diastereomeric pair separable using conventional methods such as crystallization or chromatography. Following the separation, the chiral moiety with which the compound was derivatized, may be removed.

Racemic mixtures of compounds of formula I with their corresponding (R)-enantiomers may be separated without diastereomer formation by differential absorption on a chiral stationary phase of a chromatography column, particularly a preparative HPLC column. Chiral HPLC columns are commercially available with a variety of packing materials to suit a broad range of separation applications. Exemplary stationary phases suitable for resolving the racemic 2,3-benzodiazepines include:

(i) macrocyclic glycopeptides, such as silica-bonded vancomycin which contains 18 chiral centers surrounding three pockets or cavities;

(ii) chiral α₁-acid glycoprotein;

(iii) human serum albumin; and

(iv) cellobiohydrolase (CBH).

Chiral α₁-acid glycoprotein is a highly stable protein immobilized onto spherical silica particles that tolerates high concentrations of organic solvents, high and low pH, and high temperatures. Human serum albumin, though especially suited for the resolution of weak and strong acids, zwitterionic and nonprotolytic compounds, has been used to resolve basic compounds. CBH is a very stable enzyme that has been immobilized onto spherical silica particles and is preferentially used for the separation of enantiomers of basic drugs from many compound classes.

The resolution of tofisopam by chiral chromatography using macrocyclic glycopeptide as a stationary phase on a Chirobiotic V™ column (ASTEAC, Whippany, N.J.) is disclosed in U.S. Pat. No. 6,080,736. Fitos et al. (J. Chromatogr., 709 265 (1995)), the entire disclosures of which are incorporated herein by reference, discloses another method for resolving racemic tofisopam by chiral chromatography using a chiral α₁-acid glycoprotein as a stationary phase on a CHIRAL-AGP™ column (ChromTech, Cheshire, UK). This method separates the (R)- and (S)-enantiomers and also resolves the two conformers (discussed below) of each enantiomer. These methods, may be used to separate racemic 2,3-benzodiazepines of formula I into individual (R)- and (S)-enantiomers. The Chirobiotic V™ column is available in a semi-preparative size as employed for the above separation 500 mm×10 mm). In addition, the stationary phase of the Chirobiotic V™ column is commercially available in bulk for packing of preparative chromatography columns with larger sample capacity.

In addition to existing as (R)- and (S)-enantiomers, 2,3-benzodiazepines also exist in two stable conformations that may be assumed by the benzodiazepine ring as generally depicted below.

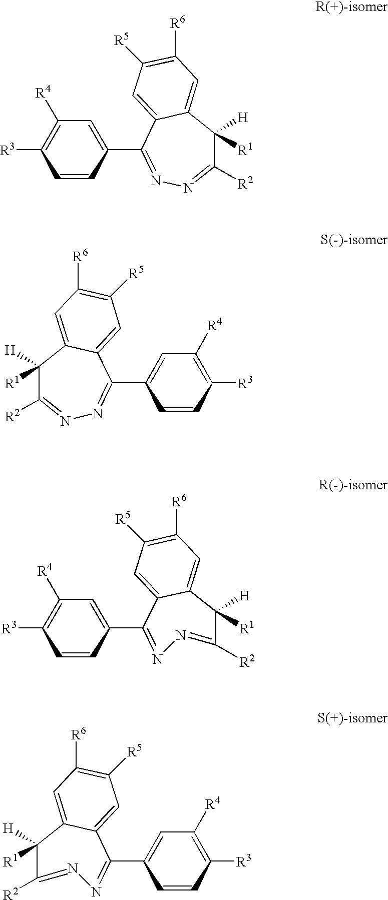

The compound used in the method of the present invention may take the form of a pharmaceutically-acceptable salt. The term “salts”, embraces salts commonly used to form alkali metal salts and to form addition salts of free acids or free bases. The term “pharmaceutically-acceptable salt” refers to salts that possess toxicity profiles within a range so as to have utility in pharmaceutical applications. Pharmaceutically unacceptable salts may nonetheless possess properties such as high crystallinity, which have utility in the practice of the present invention, such as for example utility in a synthetic process or in the process of resolving enantiomers from a racemic mixture. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, beta-hydroxybutyric, salicyclic, galactaric and galacturonic acid.

Suitable pharmaceutically acceptable base addition salts of compounds of formula I, include for example, metallic salts made from calcium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound of formula I by reacting, for example, the appropriate acid or base with the compound of formula I.

The compounds useful in the methods of the invention may be administered to individuals afflicted with dopamine-mediated disorders.

For treating dopamine-mediated disorders, the specific dose of a compound of formula I to obtain therapeutic benefit will, of course, be determined by the particular circumstances of the individual patient including, the size, weight, age and sex of the patient. Also determinative will be the nature and stage of the disease and the route of administration.

For example, a daily dosage of from about 1 to 500 mg/kg/day may be utilized. Preferably, a daily dosage of from about 5 to 300 mg/kg/day may be utilized. More preferably, a daily dosage of from about 10 to 100 mg/kg/day may be utilized.

For prophylactic administration, the compound should be administered far enough in advance of a recurrence of symptoms such that the compound is able to reach the site of action in sufficient concentration to exert a therapeutic effect. The pharmacokinetics of specific compounds may be determined by means known in the art and tissue levels of a compound in a particular individual may be determined by conventional analyses.

The compound may be administered in the form of a pharmaceutical composition comprising at least one compound of formula I in combination with a pharmaceutically acceptable carrier. The active ingredient in such formulations may comprise from 0.1 to 99.99 weight percent. By “pharmaceutically acceptable carrier” is meant any carrier, diluent or excipient that is compatible with the other ingredients of the formulation and not deleterious to the recipient.

The compound may be administered for therapeutic effect by any route, for example enteral (e.g., oral, rectal, intranasal, etc.) and parenteral administration. Parenteral administration includes, for example, intravenous, intramuscular, intraarterial, intraperitoneal, intravaginal, intravesical (e.g., into the bladder), intradermal, topical or subcutaneous administration. Also contemplated within the scope of the invention is the instillation of drug in the body of the patient in a controlled formulation, with systemic or local release of the drug to occur at a later time. For administration in the therapy of chronic disorders, the compound may optionally be localized in a depot for controlled or sustained release to the circulation, or controlled or sustained release to a local site such as for example the gastrointestinal tract or a portion thereof.

The pharmaceutically acceptable carrier is selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Ed., (1990) Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agent. Stabilizing agents, antioxidizing agents and preservatives may also be added. Suitable antioxidizing agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The compositions useful in the method of the present invention may also be formulated so as to provide slow or controlled-release of the active ingredient therein. In general, a controlled-release preparation is a composition capable of releasing the active ingredient at the required rate to maintain constant pharmacological activity for a desirable period of time. Such dosage forms may provide a supply of a drug to the body during a predetermined period of time and thus maintain drug levels in the therapeutic range for longer periods of time than other non-controlled formulations.

For example, U.S. Pat. No. 5,674,533 discloses controlled-release compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 discloses a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 discloses controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. The patents cited above are incorporated herein by reference.

Biodegradable microparticles may be used in controlled-release formulations useful in the method of this invention. For example, U.S. Pat. No. 5,354,566 discloses a controlled-release powder that contains the active ingredient. U.S. Pat. No. 5,733,566 describes the use of polymeric microparticles that release antiparasitic compositions. These patents are incorporated herein by reference.

The controlled-release of the active ingredient may be stimulated by various inducers, for example pH, temperature, enzymes, water, or other physiological conditions or compounds. Various mechanisms of drug release exist. For example, in one embodiment, the controlled-release component can swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the compound of formula I in a pharmaceutical composition. In another embodiment, the controlled-release component may be biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The practice of the invention is illustrated by the following non-limiting examples.

### EXAMPLES

### Example 1

### Synthesis of racemic-1-(3,4-dimethoxyphenyl)-4-methyl-5-ethyl-7,8-dimethoxy-5H-2,3-benzodiazepine (racemic tofisopam)

4.41 g (10 mmol) of 1-(3,4-dimethoxyphenyl)-3-methyl-4-ethyl-6,7-dimethoxyisobenzopyrilium chloride hydrochloride is dissolved in methanol (35 mL) at a temperature of 40° C. After cooling to 20-25° C., hydrazine hydrate (0.75 g, 15 mmol, dissolved in 5 mL methanol) is added. The reaction is monitored by HPLC and when complete, is evaporated to dryness. The residue is triturated with cold water (3 mL), filtered and dried to yield the crude (R,S)-1-(3,4-dimethoxyphenyl)-4-methyl-5-ethyl-7-hydroxy-8-methoxy-5H-2,3-benzodiazepine which is subsequently triturated with hot ethyl acetate to yield the pure product.

### Example 2

### Resolution of Racemic Tofisopam to Produce (S)-1-(3,4-dimethoxyphenyl)-4-methyl-5-ethyl-7-hydroxy-8-methoxy-5H-2,3-benzodiazepine((S)-tofisopam)

Racemic-1-(3,4-dimethoxyphenyl)-4-methyl-5-ethyl-7-hydroxy-8-methoxy-5H-2,3-benzodiazepine (43 mg, dissolved in acetonitrile) is injected onto a Chirobiotic V column (ASTEAC, Whippany, N.J.). Elution of the racemate with methyl-tert-butyl ether/acetonitrile 90/10 (v/v), at 40 mL/minute, is monitored at 310 nm, 2 mm path.

The (R)-(+) enantiomer is the first compound to elute, and is collected and dried. The (R)-(−), (S)-(+), (S)-(−) enantiomers, and some residual (R)-(+) enantiomer coelute and are collected in subsequent fractions.

The (S)-(−) enantiomer is isolated from fraction 2. Fraction 2 was concentrated to dryness and the residue redissolved in acetonitrile (1 mL) and injected onto a Chirobiotic V column. Peaks containing the (S)-enantiomer were shave recycled over a Chirobiotic V column two more times using as a mobile phase, methyl-tert-butylether/acetonitrile in a ration of 90/10 (v/v). A peak containing (S)-(−)-tofisopam was isolated from the third recycle, dried and stored for use in biological assays. The (S)-tofisopam isolated by this chromatographic method was assayed for enantiomeric purity by analytical chromatography on a Chiral Tech OD GH060 column (Daicel) eluted with hexane/isopropyl alcohol, 90/10 (v/v), at 25° C., monitored by UV detection at 310 nm. The (S)-tofisopam was determined to be present in 74% enantiomeric excess (e.e.), or 87% by weight.

### Example 2

### Synthesis of racemic-1-(3-hydroxy-4-methoxyphenyl)-4-methyl-5-ethyl-7,8-dimethoxy-5H-2,3-benzodiazepine

Racemic-1-(3-hydroxy-4-methoxyphenyl)-4-methyl-5-ethyl-7,8-dimethoxy-5H-2,3-benzodiazepine was synthesized according to the route of Scheme 2.

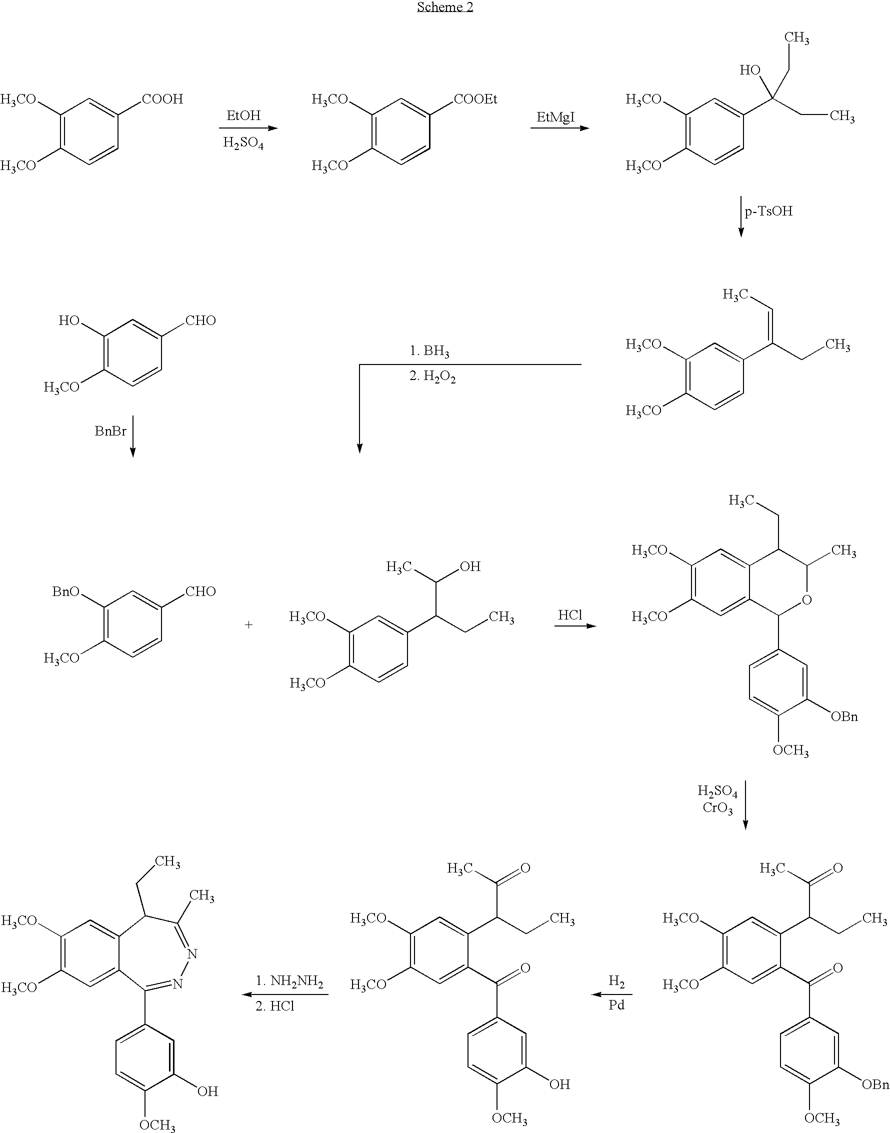

A. Esterification of 3,4-dimethoxybenzoic acid to yield ethyl-3,4-dimethoxybenzoate([3943-77-9]).

A solution of 200 g of 3,4-dimethoxybenzoic acid and 35 g of concentrated sulfuric acid in 600 mL of absolute ethanol was heated at reflux overnight. The mixture was concentrated and the residue poured into water. Methylene chloride was added and the solution washed successively with water, dilute sodium bicarbonate and water, then dried and concentrated. The residue was recrystallized from acetone/hexane.

B. Addition of ethyl magnesium iodide to ethyl-3,4-dimethoxybenzoate acid to yield 3-(3,4-dimethoxyphenyl)pentan-3-ol.

A solution of 4.8 mL of iodoethane in 20 mL of ether was added dropwise to a suspension of 1.5 g of magnesium turnings in 10 mL of ether. After 5 mL of the iodoethane solution had been added, a few grains of iodine were added and the mixture was heated to induce formation of the Grignard reagent. The remaining iodoethane solution was then added. After the Grignard formation was complete, a solution of 5 g of ethyl 3,4-dimethoxybenzoate in ether was added and the mixture was allowed to stir at room temperature overnight. The reaction was quenched by addition of saturated ammonium chloride. The mixture was extracted with ether. The combined ether extracts were dried and concentrated to an oily residue. Yield: 5 g.

C. Elimination of H₂O from 3-(3,4-dimethoxyphenyl)pentan-3-ol to yield 4-((1Z)-1-ethylprop-1-enyl)-1,2-dimethoxybenzene.

A solution of 5 g of crude 3-(3,4-dimethoxyphenyl)pentan-3-ol and 0.25 g of p-tolenesulfonic acid in 80 mL of benzene was heated at reflux for 1 hr with azeotropic removal of water. The mixture was then filtered through a pad of sodium bicarbonate and the filtrate concentrated. The residue was purified by distillation under reduced pressure. Yield: 2.9 g.

D. Addition of H₂O to 4-((1Z)-1-ethylprop-1-enyl)-1,2-dimethoxybenzene to yield 3-(3,4-dimethoxyphenyl)pentan-2-ol.

To a solution of 26 g of 4-((1Z)-1-ethylprop-1-enyl)-1,2-dimethoxybenzene in tetrahydrofuran at 0° C. was added 189 mL of a 1.0M solution of borane-tetrahydrofuran complex in tetrahydrofuran. The mixture was stirred for 3 hr at 0° C., then 35.6 mL of 50% hydrogen peroxide was added, with simultaneous addition of 5M sodium hydroxide to maintain the mixture at pH 8. The mixture was extracted with ether. The combined ether extracts were dried and concentrated.

E. Benzylation of 3-hydroxy-4-methoxybenzaldehyde to yield 4-methoxy-3-(phenylmethoxy)benzaldehyde ([6346-05-0]).

A solution of 100 g of 3-hydroxy-4-methoxybenzaldehyde and 135 g of benzyl bromide in 500 mL of acetone containing a suspension of 137 g of potassium carbonate was heated at reflux overnight. The mixture was filtered, the filtrate concentrated and the residue recrystallized from toluene/hexane. Yield: 65 g.

F. Reaction of 3-(3,4-dimethoxyphenyl)pentan-2-ol with 4-methoxy-3-(phenylmethoxy)benzaldehyde to yield 4-(4-ethyl-6,7-dimethoxy-3-methyliso-chromanyl)-1-methoxy-2-(phenylmethoxy)benzene.

A solution of 14 g of 4-methoxy-3-(phenylmethoxy)benzaldehyde and 15 g of 3-(3,4-dimethoxyphenyl)pentan-2-ol in 0.3 L of dioxane was saturated with hydrogen chloride gas. The mixture was heated at reflux for 3 hr, saturated again with hydrogen chloride gas and allowed to stir at room temperature overnight. It was then poured into water, basified with dilute sodium hydroxide and extracted with methylene chloride. The combined methylene chloride extracts were dried and concentrated.

G. Ring-opening of 4-(4-ethyl-6,7-dimethoxy-3-methyliso-chromanyl)-1-methoxy-2-(phenylmethoxy)benzene to yield 3-(4,5-dimethoxy-2-{[4-methoxy-3-(phenylmethoxy)phenyl]carbonyl}phenyl)pentan-2-one.

To a solution of 30 g of crude 4-(4-ethyl-6,7-dimethoxy-3-methyliso-chromanyl)-1-methoxy-2-(phenylmethoxy)benzene in 450 mL of acetone at 5° C. was added a solution of 30 g of chromic oxide in 300 mL of 35% sulfuric acid. The mixture was stirred at room temperature for 2 hr, neutralized by adding cold 10% sodium hydroxide and concentrated to remove acetone. Then, water was added and the mixture was extracted with methylene chloride. The combined methylene chloride extracts were dried and concentrated. The residue was purified by column chromatography on silica gel. Yield: 10 g

H. Debenzylation of 3-(4,5-dimethoxy-2-{[4-methoxy-3-(phenylmethoxy)phenyl]carbonyl}phenyl)pentan-2-one to yield 3-{2-[(3-hydroxy-4-methoxyphenyl)carbonyl]-4,5-dimethoxyphenyl}pentan-2-one.

A solution of 10 g of 3-(4,5-dimethoxy-2-{[4-methoxy-3-(phenylmethoxy)-phenyl]carbonyl}phenyl)pentan-2-one in methylene chloride containing a suspension of 0.9 g of 10% palladium on carbon was hydrogenated at 80 psi for 1 hr. The mixture was filtered through diatomaceous earth and the filtrate concentrated. Yield: 6.5 g

I. Annulation of 3-{2-[(3-hydroxy-4-methoxyphenyl)carbonyl]-4,5-dimethoxyphenyl}pentan-2-one by reaction with hydrazine to yield 1-(3-hydroxy-4-methoxyphenyl)-4-methyl-5-ethyl-7,8-dimethoxy-5H-2,3-benzodiazepine.

A solution of 6.5 g of 3-{2-[(3-hydroxy-4-methoxyphenyl)carbonyl]-4,5-dimethoxyphenyl}pentan-2-one and 2.2 mL of hydrazine in 130 mL of ethanol was heated at reflux for 0.5 hr. After allowing the solution to cool to room temperature, it was saturated with HCl gas. The mixture was then concentrated to a volume of about 5 mL, basified with concentrated ammonium hydroxide, and extracted with methylene chloride. The combined methylene chloride extracts were dried and concentrated, and the residue recrystallized from ethyl acetate/hexane. Yield: 0.97 g

The product 1-(3-hydroxy-4-methoxyphenyl)-4-methyl-5-ethyl-7,8-dimethoxy-5H-2,3-benzodiazepine was analyzed by HPLC, elemental analysis, GC/MS, proton NMR and differential scanning calorimetry (DSC). The data are as follows:

Purity: 99.29% by HPLC (% area). Column: Betasil Phenyl 4.6×150 mm. Mobile Phase: Acetonitrile:0.01M Phosphate Buffer (70::30). Flow Rate: 0.5 mL/min. Wavelength: 254 nm.

GC-MS; M/e=358; with the fragmentation pattern matching the proposed structure.

DSC: Temperature program 100° C. to 300° C. at 5° C./min, indicated molar purity=99.75% and melting point of 158.6° C.

Elemental analysis (calculated/analysis): % C, 68.09/68.08; % H, 6.61/6.57; N, 7.53/7.35. Calculated values include 0.02 equivalents of ethyl acetate and 0.09 equivalents of residual water.

NMR (DCCl₃) (performed on GE QE 300): 1.08 ppm (t, 3H); 1.99 (s, 3H); 2.11 (in, 2H); 2.75 (in, 1H); 3.75 (s, 3H); 3.93 (s, 3H); 3.97 (s, 3H); 6.46 (bs, 1H); 6.72 (s, 1H); 6.86 (m, 2H); 7.18 (d, 1H); 7.48 (s, 1H).

### Example 3

### Synthesis of 1-(3,4-dimethoxyphenyl)-4-methyl-5-ethyl-7-methoxy-8-hydroxy-5H-2,3-benzodiazepine

Racemic 1-(3,4-dimethoxyphenyl)-4-methyl-5-ethyl-7-methoxy-8-hydroxy-5H-2,3-benzodiazepine was synthesized according to the route of

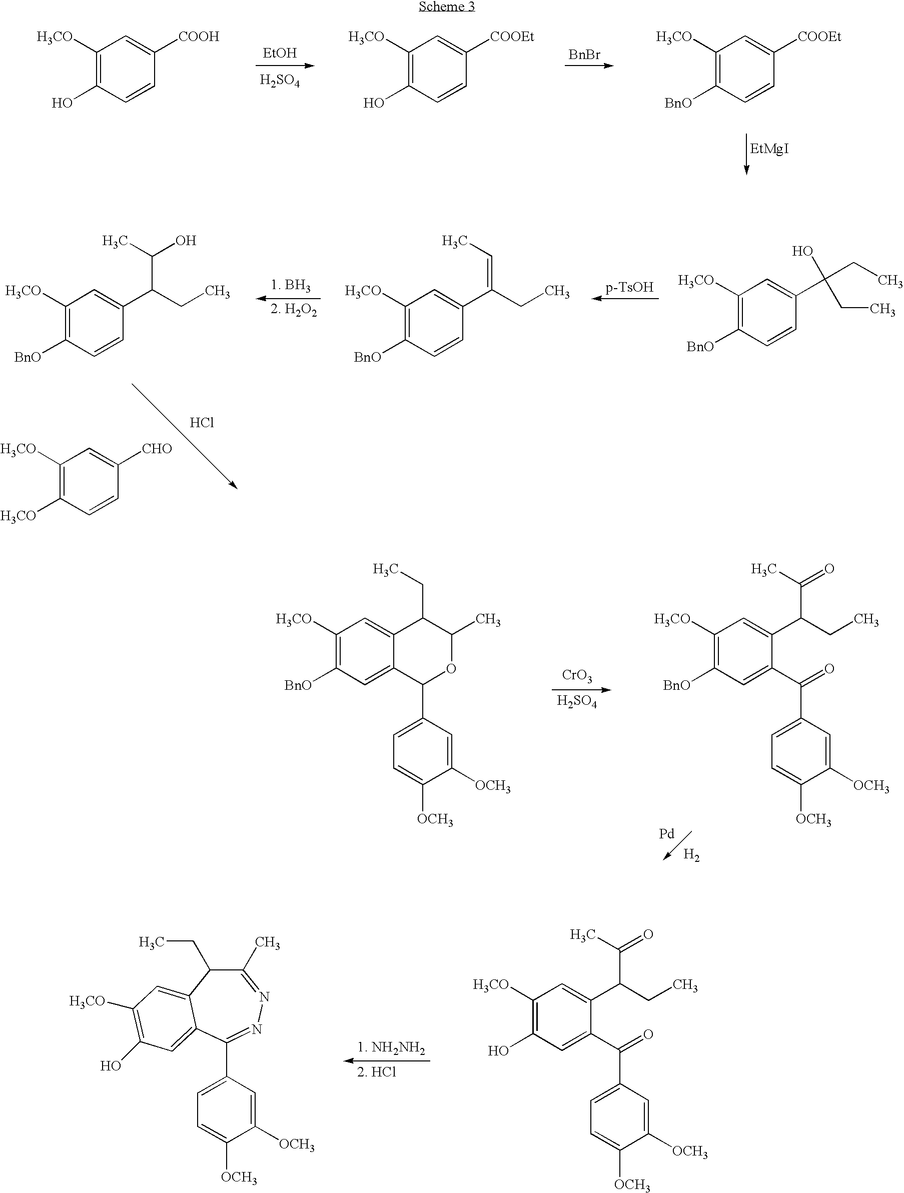

A. Esterification of 3-methoxy-4-hydroxybenzoic acid to yield ethyl-3-methoxy-4-hydroxybenzoate.

A solution of 100 g of 3-methoxy-4-hydroxybenzoic acid and 17 g of concentrated sulfuric acid in 300 mL of absolute ethanol was heated at reflux overnight. The mixture was concentrated and the residue poured into water. Methylene chloride was added and the solution washed successively with water, dilute sodium bicarbonate and water, then dried and concentrated. Yield: 118 g

B. Benzylation of ethyl-3-methoxy-4-hydroxybenzoate to yield ethyl-3-methoxy-4-benzyloxybenzoate.

A solution of 118 g of ethyl-3-methoxy-4-hydroxybenzoate and 86 mL of benzyl bromide in 600 mL of acetone containing a suspension of 124 g of potassium carbonate was heated at reflux overnight. The mixture was filtered, the filtrate concentrated and the residue recrystallized from acetone.

C. Addition of ethyl magnesium iodide to ethyl-3-methoxy-4-benzyloxybenzoate to yield 3-(3-methoxy-4-benzyloxyphenyl)pentan-3-ol.

Iodoethane (112 mL) was added dropwise to a suspension of 35 g of magnesium turnings in 160 mL of ether. After the formation of ethyl magnesium iodide was complete, a solution of 142 g of ethyl 3-methoxy-4-benzyloxybenzoate in ether was added and the mixture was allowed to stir at room temperature for 3 days. The reaction was quenched by addition of saturated ammonium chloride. The layers were separated and the ether layer was dried and concentrated to an oily residue. Yield: 110 g.

D. Elimination of H₂O from 3-(3-methoxy-4-benzyloxyphenyl)pentan-3-ol to yield 4-((1Z)-1-ethylprop-1-enyl)-1-benzyloxy-2-methoxybenzene.

A solution of 110 g of crude 3-(3-methoxy-4-benzyloxyphenyl)pentan-3-ol and 7 g of p-tolenesulfonic acid in 2 L of benzene was heated at reflux for 4 hr with azeotropic removal of water. The mixture was then filtered through a pad of sodium bicarbonate and the filtrate concentrated. The residue was purified by column chromatography on neutral alumina.

E. Addition of H₂O to 4-((1Z)-1-ethylprop-1-enyl)-1-benzyloxy-2-methoxybenzene to yield 3-(3-methoxy-4-benzyloxyphenyl)pentan-2-ol.

To a solution of 96 g of 4-((1Z)-1-ethylprop-1-enyl)-1-benzyloxy-2-methoxybenzene in tetrahydrofuran at 0° C. was added 510 mL of a 1.0M solution of borane-tetrahydrofuran complex in tetrahydrofuran. The mixture was stirred for 3 hr at 0° C., then 204 mL of 25% hydrogen peroxide was added. The mixture was adjusted to pH 8 by addition of 5M sodium hydroxide and extracted with ether. The combined ether extracts were dried and concentrated. Yield: 102 g.

F. Reaction of 3-(3-methoxy-4-benzyloxyphenyl)pentan-2-ol with 3,4-dimethoxybenzaldehyde to yield 4-(4-ethyl-6-methoxy-7-benzyloxy-3-methyliso-chromanyl)-1,2-dimethoxybenzene.

A solution of 46 g of 3,4-dimethoxybenzaldehyde and 100 g of crude 3-(3-methoxy-4-benzyloxyphenyl)pentan-2-ol in 0.3 L of dioxane was saturated with hydrogen chloride gas. The mixture was heated at reflux for 3 hr, then poured into water, basified with dilute sodium hydroxide and extracted with methylene chloride. The combined methylene chloride extracts were dried and concentrated.

G. Ring-opening of 4-(4-ethyl-6-methoxy-7-benzyloxy-3-methyliso-chromanyl)-1,2-dimethoxybenzene to yield 3-(4-benzyloxy-5-methoxy-2-{[3,4-dimethoxyphenyl]carbonyl}phenyl)pentan-2-one.

To a solution of 50 g of crude 4-(4-ethyl-6-methoxy-7-benzyloxy-3-methyliso-chromanyl)-1,2-dimethoxybenzene in acetone at 5° C. was added a solution of 50 g of chromic oxide in 500 mL of 35% sulfuric acid. The mixture was stirred at room temperature for 2 hr, neutralized by adding cold 10% sodium hydroxide and concentrated to remove acetone. Water was added and the mixture extracted with methylene chloride. The combined methylene chloride extracts were dried and concentrated. The residue was purified by column chromatography on silica gel. Yield: 18 g

H. Debenzylation of 3-(4-benzyloxy-5-methoxy-2-{[3,4-dimethoxyphenyl]carbonyl}phenyl)pentan-2-one to yield 3-{2-[(3,4-dimethoxyphenyl)carbonyl]-4-hydroxy-5-methoxyphenyl}pentan-2-one.

A solution of 18 g of 3-(4-benzyloxy-5-methoxy-2-{[3,4-dimethoxyphenyl]carbonyl}phenyl)pentan-2-one in methylene chloride containing a suspension of 2 g of 10% palladium on carbon was hydrogenated at 80 psi for 1 hr. The mixture was filtered through diatomaceous earth and the filtrate concentrated. Yield: 15 g

I. Annulation of 3-{2-[(3,4-dimethoxy-phenyl)carbonyl]-4-hydroxy-5-methoxyphenyl}pentan-2-one by reaction with hydrazine to yield 1-(3,4-dimethoxyphenyl)-4-methyl-5-ethyl-7-methoxy-8-hydroxy-5H-2,3-benzodiazepine.

A solution of 14 g of 3-{2-[(3,4-dimethoxy-phenyl)carbonyl]-4-hydroxy-5-methoxyphenyl}pentan-2-one and 4.7 mL of hydrazine in 280 mL of ethanol was heated at reflux for 0.5 hr. After allowing the solution to cool to room temperature, it was saturated with HCl gas. The mixture was then concentrated to a volume of about 5 mL, basified with concentrated ammonium hydroxide, and extracted with methylene chloride. The combined methylene chloride extracts were dried and concentrated, and the residue recrystallized from ethyl acetate/hexane. Yield: 1.5 g

The product 1-(3,4-dimethoxyphenyl)-4-methyl-5-ethyl-7-methoxy-8-hydroxy-5H-2,3-benzodiazepine was analyzed by HPLC, elemental analysis, GC/MS, proton NMR and differential scanning calorimetry (DSC). The data are as follows:

Purity: 98.36% by HPLC (% area). Column: Betasil Phenyl 4.6×150 mm. Mobile Phase: Acetonitrile::0.01M Phosphate Buffer (70::30). Flow Rate: 0.5 mL/min. Wavelength: 254 nm.

GC-MS; M/e=358; with the fragmentation pattern matching the proposed structure.

Differential scanning calorimetry (DSC): Temperature program 100° C. to 300° C. at 5° C./min, indicated molar purity=99.14% and melting point of 146.2° C.

Elemental analysis (calculated/analysis): % C, 68.14/68.12; % H, 6.63/6.63; N, 7.43/7.20. The calculated values include 0.1M of residual ethyl acetate.

NMR (DCCl₃) (performed on GE QE 300): 1.08 ppm (t, 3H); 1.96 (s, 3H); 2.10 (m, 2H); 2.77 (m, 1H); 3.91 (s, 3H); 3.93 (s, 3H); 3.98 (s, 3H); 5.73 (bs, 1H); 6.70 (s, 1H); 6.80 (d, 1H); 6.95 (s, 1H); 7.00 (d, 1H); 7.58 (s, 1H).

### Example 4

### Resolution of 1-(3-hydroxy-4-methoxyphenyl)-4-methyl-5-ethyl-7,8-dimethoxy-5H-2,3-benzodiazepine

The enantiomers of racemic-1-(3-hydroxy-4-methoxyphenyl)-4-methyl-5-ethyl-7,8-dimethoxy-5H-2,3-benzodiazepine are resolved by chiral chromatography as follows.

Racemic-1-(3-hydroxy-4-methoxyphenyl)-4-methyl-5-ethyl-7,8-dimethoxy-5H-2,3-benzodiazepine is loaded onto a semipreparative (500 mm×10 mm) Chirobiotic V column (ASTEC, Whippany, N.J.). Elution of the enantiomeric mixture with methyl-tert-butyl ether/acetonitrile (90/10 V/V), at a flow rate of 40 mL/min, is monitored at 310 nm. Fraction size is 10-20 mL and fractions are subjected to analytical chromatography using the same solvent composition on an analytical (150×4.6 mm) Chirobiotic V column. The fractions containing each isolated enantiomer are processed by removing the elution solvent in vacuo.

### Example 5

### Low Dose Apomorphine Test in the Mouse

Racemic tofisopam, (R)-tofisopam and (S)-tofisopam were investigated in the Low Dose Apomorphine Test in the mouse to evaluate their pro- or anti-dopaminergic activity following intraperitoneal (i.p.) administration of apomorphine. Apomorphine, at a low dose, induces hypothermia, stereotypies and climbing. All three of these symptoms are potentiated by dopaminergic agonists and antagonized by dopamine antagonists, such as classical neuroleptics. The Low Dose Apomorphine Test, thus serves to detect pro- or antidopaminergic activity. The test was performed according to the protocols described by Chermat and Poncelet. See, Chermat R., Poncelet M., “Antagonisme des redressements, des stéréotypies et de l'hypotheimie induits par 1 ou 16 mg.kg-1 d'apomorphine,” J. Pharmacol., 14, 93-97, 1983.

A. Animals and General Study Procedure

Male mice of Rj: NMRI strain, 23-26 g body weight range were supplied by Elevage Janvier, 53940 Le Genest-Saint-Isle, France. The animals were delivered to the laboratory at least 3 days before the experiment and, upon arrival, were housed 10 per cage in macrolon cages (25×19×13 cm) containing wood shavings (Litalabo-SPPS, 95100 Argenteuil, France) with free access to food (UAR 113-UAR, 91360 Epinay-sur-Orge, France) and tap water (i.e. non-fasted) until tested. The animal house was maintained under artificial lighting (12 hours) between 7.00 and 19.00 in a controlled ambient temperature of 21±1° C., and relative humidity maintained at 40-70%.

The different treatments were equally distributed between the animals. During the experiment, each animal within a cage received the same treatment. Animals within a cage were individually marked on the tail with an indelible pen.

The experiment was conducted blind with treatment vials labeled A, B, C etc., according to a standardized coding procedure. The evaluation of the different treatments was balanced over time by testing successive series of individual animals from each treatment group in a fixed rotation (A, B, C, etc.). Animals were sacrificed at the end of the experiment by exposure to CO₂.

B. Test Substances Administered.

Three compounds, racemic tofisopam, (R)-tofisopam and (S)-tofisopam were tested in the Low Dose Apomorphine Test. Each of these three compounds was dosed as a dispersion in 0.2% hydroxypropylmethylcellulose (HPMC) (Sigma), in sterile physiological saline (Laboratoire Aguettant).

Two reference standards were also dosed. Bromocriptine, methanesulfonate (Sigma), a known dopamine agonist, and haloperidol (Sigma), a known dopamine antagonist, were dispersed in 0.2% HPMC in sterile physiological saline.

Apomorphine hydrochloride, the dopamine mimetic employed in the assay, was dissolved in water for injectable preparation (Laboratoire Aguettant). Dosing: All of the dosed substances were administered in a volume of 10 ml/kg. The doses are expressed in mg/kg of supplied substance.

C: Procedure

6 mice were studied per group. The test was performed blind.

Racemic tofisopam, (R)-tofisopam and (S)-tofisopam were administered at doses of 16, 32 and 64 mg/kg, administered intraperitonealy (i.p.), 30 minutes prior to administration of apomorphine.

The rectal temperature of the mice was measured prior to apomorphine dosing using a rectal probe (Physitemp Instruments Digital Laboratory Thermometer Model BAT-12). The mice were then placed in individual Plexiglas cages (10×6×4 cm) attached with the opening against a vertical wire grid.

The mice were then injected subcutaneously (s.c.) with apomorphine (1 mg/kg). Rectal temperature was measured 30 and 60 minutes after the injection of apomorphine.

The intensity of stereotypies (sniffing, licking, gnawing), were scored at 20 minutes and 50 minutes after injection of apomorphine on a 4 point scale (0-3). The presence (1) or absence (0) of climbing against the wire grid wall, were also assessed 20 and 50 minutes after injection of apomorphine.

The reference standards bromocriptine (16 mg/kg) and haloperidol (0.5 mg/kg), were administered under the same experimental conditions.

The temperature measurement data, showing the changes in measured temperature during the Low Dose Apomorphine Assay is listed in Table 1. Dosing of vehicle alone provides a control that shows the hypothermia effect of apomorphine with no drug administered subsequently.

TABLE 1

| Test | Rectal Temperature (° C.) | | | | | | | | |
|---|---|---|---|---|---|---|---|---|---|
| substance | | 30 min after apomorphine | | | | 60 min after apomorphine | | | |
| Dose | Before | | | p | mean change | | | p | mean change |
| (mg/kg) | apomorphine | mean ± s.e.m | | value | from control | mean ± s.e.m. | | value | from control |
| Vehicle | 37.3 ± 0.1 | 35.1 ± 0.3 | — | — | — | 36.7 ± 0.2 | | — | — |
| Racemic | 37.3 ± 0.1 | 35.3 ± 0.6 | NS | 0.823 | +0.2 | 36.9 ± 0.3 | NS | 0.532 | +0.2 |
| tofisopam | | | | | | | | | |
| 16 mg/kg | | | | | | | | | |
| Racemic | 37.1 ± 0.2 | 34.3 ± 0.5 | NS | 0.164 | −0.8 | 36.6 ± 0.3 | NS | 0.728 | −0.1 |
| tofisopam | | | | | | | | | |
| 32 mg/kg | | | | | | | | | |
| Racemic | 37.2 ± 0.2 | 33.9 ± 0.5 | NS | 0.066 | −1.2 | 36.3 ± 0.3 | NS | 0.177 | −0.4 |
| tofisopam | | | | | | | | | |
| 64 mg/kg | | | | | | | | | |
| (S)-tofisopam | 37.2 ± 0.3 | 34.8 ± 0.4 | NS | 0.529 | −0.3 | 37.2 ± 0.2 | * | 0.046 | +0.5 |
| 16 mg/kg | | | | | | | | | |
| (S)-tofisopam | 37.0 ± 0.2 | 34.4 ± 0.3 | NS | 0.089 | −0.7 | 36.6 ± 0.2 | NS | 0.732 | −0.1 |
| 32 mg/kg | | | | | | | | | |
| (S)-tofisopam | 37.2 ± 0.2 | 33.6 ± 0.7 | NS | 0.066 | −1.5 | 33.3 ± 1.0 | ** | 0.009 | −3.4 |
| 64 mg/kg | | | | | | | | | |
| (R)-tofisopam | 37.3 ± 0.1 | 35.1 ± 0.3 | NS | 0.968 | 0.0 | 36.9 ± 0.2 | NS | 0.347 | +0.2 |
| 16 mg/kg | | | | | | | | | |
| (R)-tofisopam | 37.1 ± 0.1 | 34.6 ± 0.4 | NS | 0.270 | −0.5 | 36.6 ± 0.2 | NS | 0.781 | −0.1 |
| 32 mg/kg | | | | | | | | | |
| (R)-tofisopam | 37.2 ± 0.1 | 35.1 ± 0.4 | NS | 0.973 | 0.0 | 36.5 ± 0.4 | NS | 0.691 | −0.2 |
| 64 mg/kg | | | | | | | | | |
| Bromocriptine | 37.2 ± 0.2 | 34.4 ± 0.4 | NS | 0.181 | −0.7 | 35.0 ± 0.5 | ** | 0.007 | −1.7 |
| 16 mg/kg | | | | | | | | | |
| Haloperidol | 37.3 ± 0.1 | 37.0 ± 0.1 | *** | 0.000 | +1.9 | 37.7 ± 0.1 | *** | 0.000 | +1.0 |
| 0.5 mg/kg | | | | | | | | | |

Student's t test:
NS = Not Significant;
* = p < 0.05;
** = p < 0.01;
*** = p < 0.001
s.e.m. = standard error of the mean.

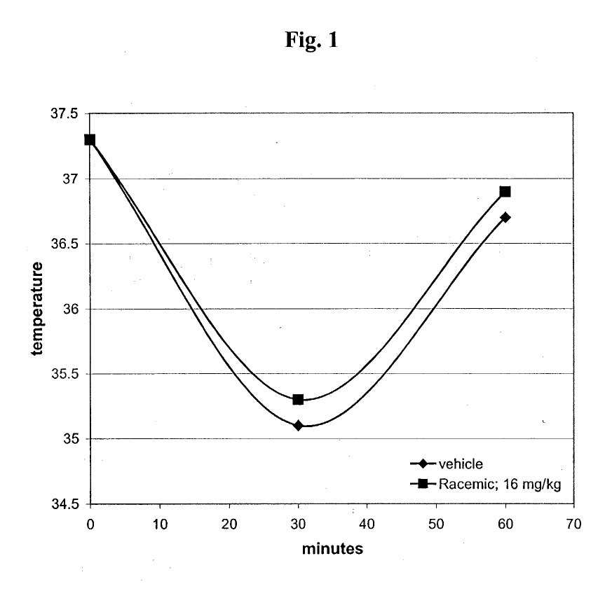

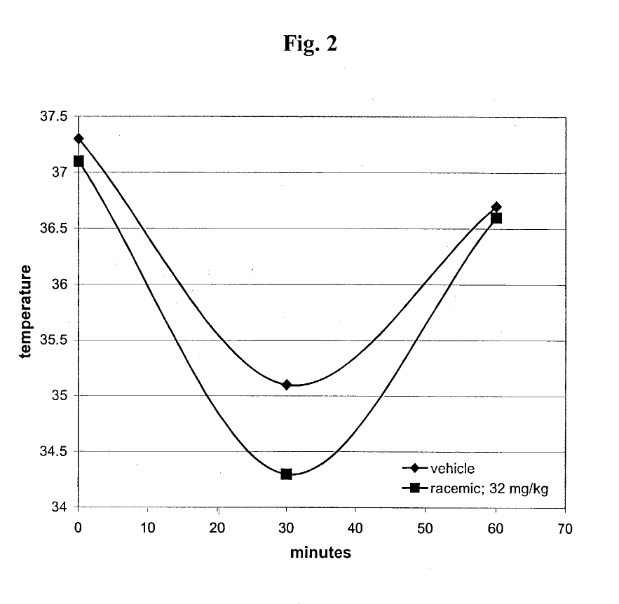

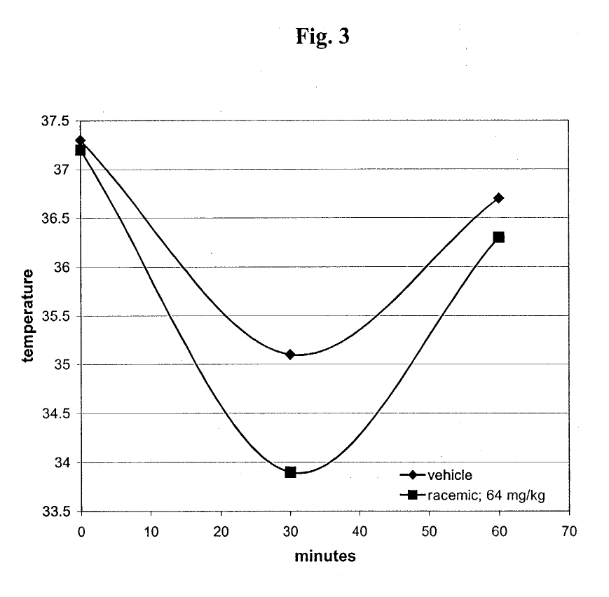

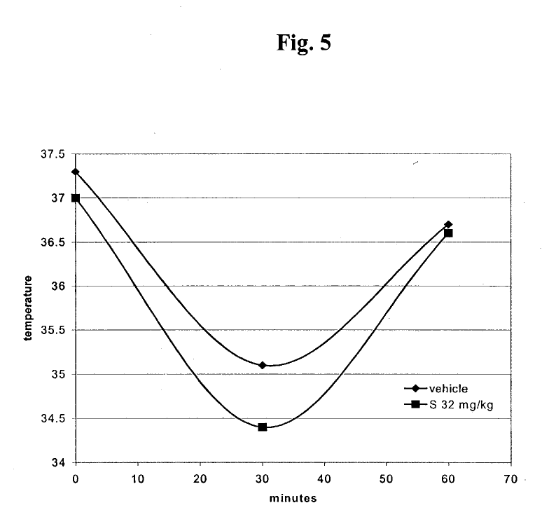

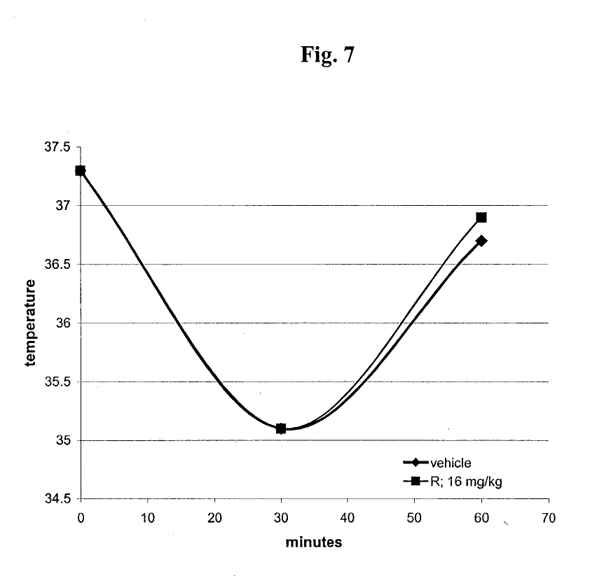

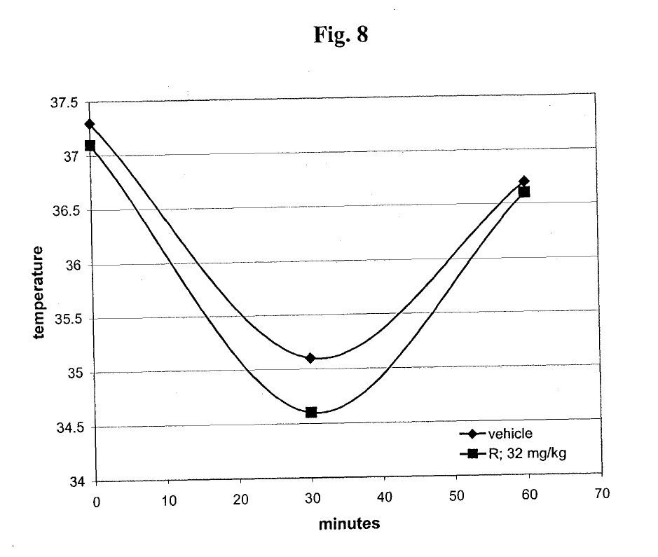

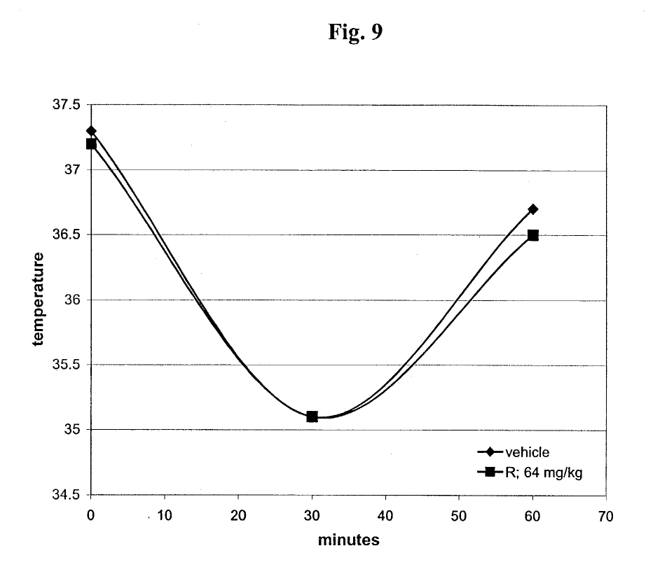

The temperature data listed in Table 1 is plotted graphically in FIGS. 1-11. The data show (See, FIGS. 7, 8 and 9) that (R)-tofisopam did not affect apomorphine-induced hypothermia. Racemic tofisopam at 64 mg/kg tended to behave as a weak dopamine antagonist, i.e., lowering the rectal temperature at the thirty and sixty minute time points (FIGS. 1, 2, and 3), however this trend was not statistically significant.

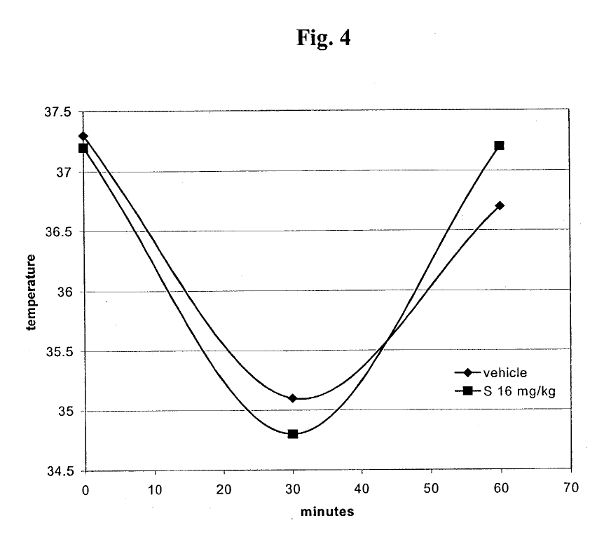

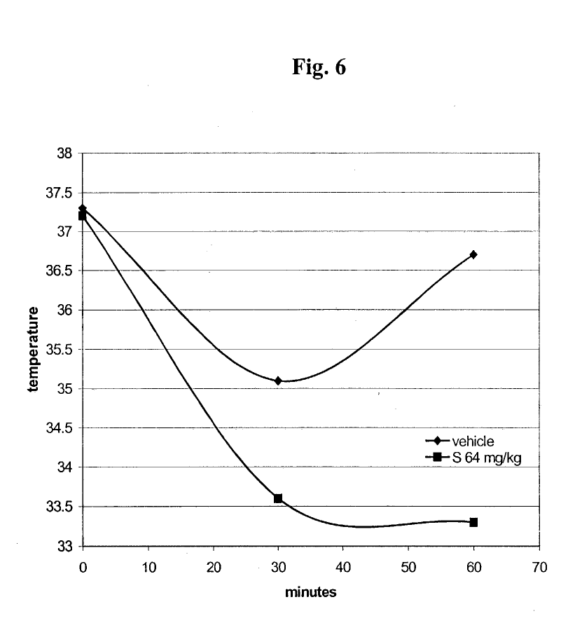

The data show that (S)-tofisopam behaved as a weak dopamine antagonist at the 16 mg/kg dose at sixty minutes after apomorphine administration, i.e., showing a slight but statistically significant elevation in temperature. At the higher doses, (S)-tofisopam demonstrated dopamine antagonism at both the thirty minute and sixty minute time points, i.e., lowering the rectal temperature at both time points (FIGS. 4, 5 and 6). This effect was both large and statistically significant at a dose of 64 mg/kg at 60 minutes after apomorphine (FIG. 6).

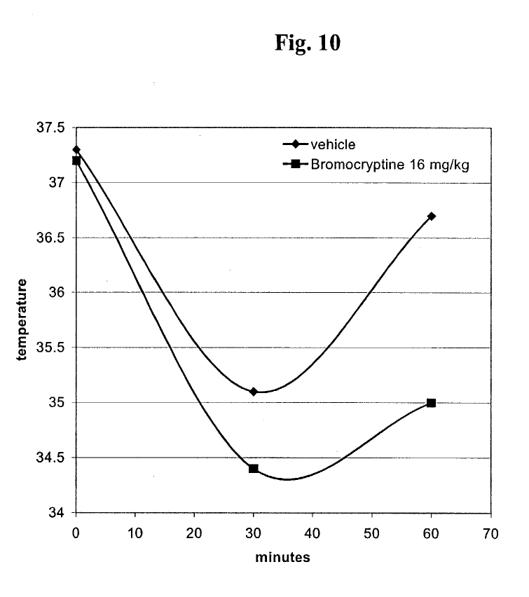

The dopamine agonist, bromocriptine (16 mg/kg), administered under the same experimental conditions, significantly potentiated apomorphine-induced hypothermia, as shown in FIG. 10.

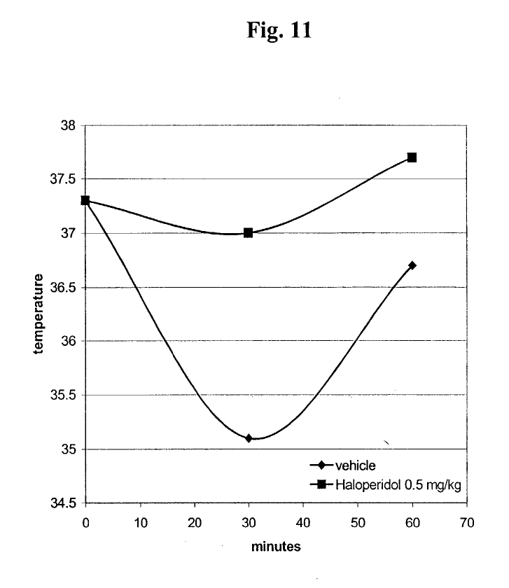

In contrast, dopamine antagonist, haloperidol (0.5 mg/kg), significantly antagonized apomorphine-induced hypothermia, elevating rectal temperature at 60 minutes as seen in FIG. 11.

The data for the scoring of apomorphine-induced stereotypies and climbing is listed in Table 2.

TABLE 2

| | STEREOTYPY | | | | | | | | CLIMBING | |
|---|---|---|---|---|---|---|---|---|---|---|
| Test | (intensity score) | | | | | | | | (No. of mice) | |
| substance | 20 min after apomorphine | | | | 50 min after apomorphine | | | | minutes after | |
| Dose | | | p | mean change | | | p | mean change | apomorphine | |
| (mg/kg) | mean ± s.e.m. | | value | from control | mean ± s.e.m. | | value | from control | 20 | 50 |
| Vehicle | 2.2 ± 0.3 | | — | — | 1.0 ± 0.3 | | — | — | 5 | 3 |
| Racemic | 3.0 ± 0.0 | * | 0.021 | +0.8 | 0.7 ± 0.2 | NS | 0.336 | −0.3 | 6 | 3 |
| tofisopam | | | | | | | | | | |
| 16 mg/kg | | | | | | | | | | |
| Racemic | 2.8 ± 0.2 | NS | 0.083 | +0.6 | 0.8 ± 0.3 | NS | 0.652 | −0.2 | 6 | 5 |
| tofisopam | | | | | | | | | | |
| 32 mg/kg | | | | | | | | | | |
| Racemic | 2.3 ± 0.2 | NS | 0.715 | +0.1 | 1.3 ± 0.2 | NS | 0.336 | +0.3 | 6 | 4 |
| tofisopam | | | | | | | | | | |
| 64 mg/kg | | | | | | | | | | |
| (S)-tofisopam | 3.0 ± 0.0 | * | 0.021 | +0.8 | 0.7 ± 0.2 | NS | 0.336 | −0.3 | 6 | 3 |
| 16 mg/kg | | | | | | | | | | |
| (S)-tofisopam | 2.3 ± 0.3 | NS | 0.665 | +0.1 | 0.3 ± 0.2 | NS | 0.075 | −0.7 | 6 | 2 |
| 32 mg/kg | | | | | | | | | | |
| (S)-tofisopam | 0.2 ± 0.2 | ** | 0.003 | −2.0 | 0.2 ± 0.2 | * | 0.026 | −0.8 | 0+ | 0 |
| 64 mg/kg | | | | | | | | | | |
| (R)-tofisopam | 2.8 ± 0.2 | NS | 0.083 | +0.6 | 1.0 ± 0.3 | NS | 1.000 | 0.0 | 6 | 5 |
| 16 mg/kg | | | | | | | | | | |
| (R)-tofisopam | 2.2 ± 0.2 | NS | 0.923 | 0.0 | 1.0 ± 0.3 | NS | 1.000 | 0.0 | 6 | 4 |
| 32 mg/kg | | | | | | | | | | |
| (R)-tofisopam | 1.5 ± 0.6 | NS | 0.405 | −0.7 | 1.2 ± 0.3 | NS | 0.652 | +0.2 | 3 | 2 |
| 64 mg/kg | | | | | | | | | | |
| Bromocriptine | 2.7 ± 0.2 | NS | 0.212 | +0.5 | 1.0 ± 0.4 | NS | 1.000 | 0.0 | 6 | 2 |
| 16 mg/kg | | | | | | | | | | |
| Haloperidol | 0.0 ± 0.0 | ** | 0.002 | −2.2 | 0.0 ± 0.0 | ** | 0.006 | −1.0 | 0+ | 1 |
| 0.5 mg/kg | | | | | | | | | | |

Mann-Whitney U-test (stereotypies):
NS = Not Significant;
* = p < 0.05;
** = p < 0.01
Fisher's Exact test (climbing): no indication = not significant; + = p < 0.05
s.e.m. = standard error of the mean.

The data in Table 2 show that racemic tofisopam at low doses tended to behave as a strong dopamine agonist, i.e., increasing the stereotypies induced by apomorphine at the 16 and 32 mg/kg doses at the twenty minute time point, though the increase was both strong and statistically significant only at the twenty minute time point for the 16 mg/kg dose. (S)-tofisopam also behaved as a strong dopamine agonist at the 16 mg/kg dose at the twenty minute time point. However, unlike racemic tofisopam, (S)-tofisopam, behaved as a strong dopamine antagonist at the 64 mg/kg dose, i.e., showing a statistically significant decrease in apomorphine-induced stereotypies and climbing at both the twenty minute and the fifty minute time points.

(R)-tofisopam did not affect apomorphine-induced hypothermia, stereotypies or climbing in the dose-range tested.

The reference standard bromocriptine (16 mg/kg), administered under the same experimental conditions, did not significantly affect apomorphine-induced stereotypies or climbing even though it potentiated apomorphine-induced hypothermia.

Haloperidol (0.5 mg/kg) significantly antagonized apomorphine-induced stereotypies and climbing as well as hypothermia.

Thus, the temperature data and the stereotypy data from the Low Dose Apomorphine assay show that compounds of formula I are useful in modulation of dopamine responses and in the treatment of dopamine-mediated disorders.

All references cited herein are incorporated by reference. The present invention may be embodied in other specific forms without departing from the spirit or essential attributes thereof and, accordingly, reference should be made to the appended claims, rather than to the foregoing specification, as indication the scope of the invention.

What is claimed is:

1. A method of modulating dopamine responses in the central nervous system of an individual, said method comprising administering to the individual an effective amount of at least one compound of formula I: 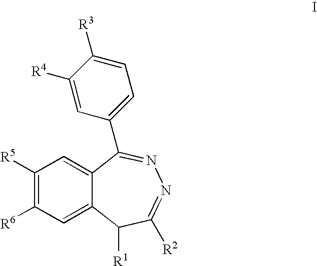
wherein:
R¹ is —(C₁-C₇)hydrocarbyl or —(C₂-C₆)heteroalkyl;
R² is —H or —(C₁-C₇)hydrocarbyl; wherein R¹ and R² may combine to form a carbocyclic or heterocyclic 5- or 6-membered ring; and
R³, R⁴, R⁵ and R⁶ are independently selected from the group consisting of —OH, —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen, wherein R⁵ and R⁶ may combine to form a 5,6- or 7-membered heterocyclic ring;
or a pharmaceutically acceptable salt thereof;
said compound comprising an (S)-enantiomer substantially free of the (R)-enantiomer of the same compound.

2. A method of treating a dopamine-mediated disorder in an individual not suffering from seizures or convulsions, said method comprising administering to the individual an effective amount of at least one compound of formula I: 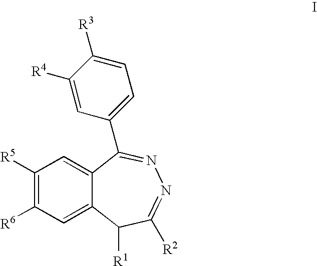
wherein:
R¹ is —(C₁-C₇)hydrocarbyl or —(C₂-C₆)heteroalkyl;
R² is —H or —(C₁-C₇)hydrocarbyl; wherein R¹ and R² may combine to form a carbocyclic or heterocyclic 5- or 6-membered ring; and
R³, R⁴, R⁵ and R⁶ are independently selected from the group consisting of —OH, —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen, wherein R⁵ and R⁶ may combine to form a 5,6- or 7-membered heterocyclic ring;
or a pharmaceutically acceptable salt thereof;
said compound comprising an (S)-enantiomer substantially free of the (R)-enantiomer of the same compound.

3. The method according to claim 2, wherein the dopamine-mediated disorder comprises a neurological disorder or a neuropsychiatric disorder.

4. The method according to claim 3 wherein the neurological disorder is selected from the group consisting of Huntington's chorea, Parkinson's disease, periodic limb movement syndrome, restless leg syndrome, hyperkinesias, Tourette's syndrome, Pick's disease, punch drunk syndrome, progressive subnuclear palsy, multiple systems atrophy, Landau-Kleffner syndrome, benign essential blepharospasm, amyotrophic lateral sclerosis, medication-induced movement disorders, and cognitive disorders.

5. The method according to claim 4 wherein the medication-induced movement disorder is selected from the group consisting of neuroleptic-induced Parkinsonism, neuroleptic malignant syndrome, acute dystonia and extrapyramidal effects of neuroleptic agents.

6. The method according to claim 4 wherein the cognitive disorder is selected from the group consisting of learning disorders, memory disorders, Alzheimer's Disease, and dementia.

7. The method according to claim 6 wherein the dementia is selected from the group consisting of pseudo dementia, hydrocephalic dementia, subcortical dementia, and dementia secondary to Huntington's chorea or Parkinson's disease.

8. The method according to claim 3 wherein the neuropsychiatric disorder is selected from the group consisting of psychosis, personality disorders, psychiatric mood disorders, conduct and impulse disorders, schizophrenia, bipolar disorders, dysphoric mania, anxiety disorders, depression, panic disorders, agoraphobia, obsessive-compulsive disorders and eating disorders.

9. The method according to claim 8 wherein the eating disorder is anorexia cachexia or anorexia nervosa.

10. The method according to claim 8 wherein the anxiety disorder is selected from the group consisting of post traumatic stress disorder, acute stress disorder, social anxiety disorder and generalized anxiety disorder.

11. A method of treating a dopamine-mediated disorder in an individual, said method comprising administering to the individual an effective amount of at least one compound of formula I: 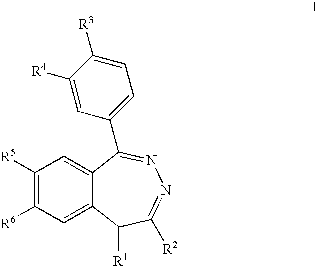
wherein:
R¹ is —(C₁-C₇)hydrocarbyl or —(C₂-C₆)heteroalkyl;
R² is —H or —(C₁-C₇)hydrocarbyl; wherein R¹ and R² may combine to form a carbocyclic or heterocyclic 5- or 6-membered ring; and
R³, R⁴, R⁵ and R⁶ are independently selected from the group consisting of —OH, —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen, wherein R⁵ and R⁶ may combine to form a 5,6- or 7-membered heterocyclic ring;
or a pharmaceutically acceptable salt thereof;
said compound comprising an (S)-enantiomer substantially free of the (R)-enantiomer of the same compound.
provided that the dopamine-mediated disorder is not one which causes seizures or convulsions.

12. The method according to claim 11 wherein the dopamine-mediated disorder comprises a neurological disorder selected from the group consisting of periodic limb movement syndrome, restless leg syndrome, hyperkinesias, punch drunk syndrome, progressive subnuclear palsy, multiple systems atrophy, Landau-Kleffner syndrome, benign essential blepharospasm, medication-induced movement disorders and cognitive disorders.

13. The method according to claim 12 wherein the medication-induced movement disorder is selected from the group consisting of neuroleptic malignant syndrome, acute dystonia and extrapyramidal effects of neuroleptic agents.

14. The method according to claim 12 wherein the cognitive disorder is selected from the group consisting of learning disorders, memory disorders and dementia.

15. The method according to claim 14 wherein the dementia is selected from the group consisting of pseudo dementia, hydrocephalic dementia and subcortical dementia.

16. The method according to claim 11 wherein the dopamine-mediated disorder comprises a neuropsychiatric disorder selected from the group consisting of psychosis, personality disorders, psychiatric mood disorders, conduct and impulse disorders, bipolar disorders, dysphoric mania, attention deficit hyperactivity disorders, anxiety disorders, depression, panic disorders, panic attack, agoraphobia and eating disorders.

17. The method according to claim 16 wherein the eating disorder is anorexia cachexia or anorexia nervosa.

18. The method according to claim 16 wherein the anxiety disorder is selected from the group consisting of post traumatic stress disorder, acute stress disorder, social anxiety disorder and generalized anxiety disorder.

19. The method of claim 2 or claim 10 wherein each of the group R³, R⁴, R⁵ and R⁶ is independently selected from —O(C₁-C₇)hydrocarbyl.

20. The method of claim 19 wherein each of the group R³, R⁴, R⁵ and R⁶ is independently selected from —O(C₁-C₇)alkyl.

21. The method of claim 20 wherein each of the group R³, R⁴, R⁵ and R⁶ is —OCH₃.

22. The method of claim 21 wherein the compound of formula I is (S)-1-(3,4-dimethoxyphenyl)-4-methyl-5-ethyl-7,8-dimethoxy-5H-2,3-benzodiazepine, or a pharmaceutically acceptable salt thereof.

23. The method of claim 2 or claim 10 wherein one member of the group R³, R⁴, R⁵ or R⁶ is —OH and the remaining members of the group R³, R⁴, R⁵ and R⁶ are independently selected from the group consisting of —(C₁-C₇)hydrocarbyl, —CF₃, —O(C₁-C₇)hydrocarbyl, —O-acyl, —NH₂, —NH(C₁-C₆)alkyl, —N((C₁-C₆)alkyl)₂, —NH-acyl and halogen.

24. The method of claim 23 wherein R³ or R⁴ is —OH.

25. The method of claim 23 wherein the remaining members of the group R³, R⁴, R⁵ and R⁶ are independently selected from the group consisting of —O(C₁-C₇)hydrocarbyl.

26. The method of claim 25 wherein R³ or R⁴ is —OH.

27. The method of claim 25 wherein said remaining members of the group R³, R⁴, R⁵ and R⁶ are independently selected from —O(C₁-C₇)alkyl.

28. The method of claim 27 wherein R³ or R⁴ is —OH.

29. The method of claim 2 or claim 10 wherein one member of the group R³, R⁴, R⁵ and R⁶ is —OH and the remaining members of the group R³, R⁴, R⁵ and R⁶ are —OCH₃.

30. The method of claim 29 wherein R³ or R⁴ is —OH.

31. The method of claim 30 wherein R¹ and R² are independently selected from —(C₁-C₇)alkyl.

32. The method of claim 31 wherein R¹ and R² are independently selected from —(C₁-C₃)alkyl.

33. The method of claim 32 wherein R¹ is —CH₂CH₃ and R² is —CH₃.

34. The method of claim 33 wherein the compound of formula I is selected from the group consisting of:
(S)-1-(3,4-dimethoxyphenyl)-4-methyl-5-ethyl-7-hydroxy-8-methoxy-5H-2,3-benzodiazepine;
(S)-1-(3-hydroxy-4-methoxyphenyl)-4-methyl-5-ethyl-7,8-dimethoxy-5H-2,3-benzodiazepine;
(S)-1-(3-methoxy-4-hydroxyphenyl)-4-methyl-5-ethyl-7,8-dimethoxy-5H-2,3-benzodiazepine; and
(S)-1-(3,4-dimethoxyphenyl)-4-methyl-5-ethyl-7-methoxy-8-hydroxy-5H-2,3-benzodiazepine;
or a pharmaceutically acceptable salt thereof.
